(19) United States
(12) Patent Application Publication
(10) Pub. No.: US 2021/0393853 A1
(43) Pub. Date: Dec. 23, 2021

(54) COMPOSITIONS AND METHODS FOR CRYOPRESERVATION AND RECONSTITUTION OF ENGINEERED TISSUES

(71) Applicant: Avery Therapeutics, Inc., Tucson, AZ (US)

(72) Inventors: Jordan J. Lancaster, Tucson, AZ (US); Jennifer Watson Koevary, Tucson, AZ (US); Steven Goldman, Tucson, AZ (US)

(21) Appl. No.: 17/290,110

(22) PCT Filed: Oct. 29, 2019
(86) PCT No.: PCT/US19/58491

§ 371 Date: Apr. 29, 2021

Related U.S. Application Data
(60) Provisional application No. 62/752,235, filed on Oct. 29, 2018.

(51) Int. Cl. A61L 27/38; A61L 27/36; A61L 27/58; A01N 1/02

(52) U.S. Cl. CPC A61L 27/38; A01N 1/0231; A61L 27/58; A61L 27/3633

(57) ABSTRACT

Systems, methods, and compositions for cryopreservation and reconstitution of engineered tissues. The engineered tissue compositions are functional after a cryopreservation-thaw cycle For example, the tissue compositions retain their structural integrity, maintain cell-cell communication, etc. In certain embodiments, the tissue compositions exhibit synchronous contractions, secrete a hormone, secrete a cytokine, secrete an RNA, secrete a growth factor, secrete an enzyme, and/or secrete a neurotransmitter, etc. in some embodiments, engineered tissue compositions are cultured, cryopreserved, and reconstituted in a closed system.

### CROSS-REFERENCES TO RELATED APPLICATIONS

This application claims benefit of U.S. Patent Application No. 62/752,235 filed Oct. 29, 2018, the specification(s) of which is/are incorporated herein in their entirety by reference.

### FIELD OF THE INVENTION

The present invention relates to tissue engineering, e.g., three-dimensional engineered tissue compositions for allowing growth, differentiation, and/or maintenance of one or more cell types. More particularly, the present invention relates to systems (e.g., vessels, containers, etc.), compositions, and methods for cryopreservation and reconstitution of engineered tissues.

### BACKGROUND OF THE INVENTION

In recent years, three-dimensional cultures have been increasingly used to provide conditions similar to what would be expected in vivo (e.g., an appropriate structure and microenvironment) for cell growth, differentiation, and/or maintenance. A great deal of effort is currently focused on developing three-dimensional cultures that mimic specific tissues. Such three-dimensional tissue cultures may be used for a variety of purposes, such as for therapeutic purposes, for generating biological models for research and testing, etc.

### SUMMARY OF THE INVENTION

The present invention features engineered tissue compositions, e.g., three-dimensional tissue compositions that support cell growth, maintenance, and/or differentiation, etc., as well as systems (e.g., vessels, containers, etc.), compositions, and methods for cryopreservation and reconstitution of engineered tissue compositions. The systems, methods, and compositions may be applied to closed culture systems. In some embodiments, the systems, methods, and compositions may be applied to open culture systems.

As used herein, the term “open system” refers to a culture system where an operator is directly handling and opening culture plates and culture solutions/mediums. As used herein, the term “closed system” refers to a system where the cultures and solutions/mediums are fully contained in their own system and isolated from or not in contact with the surrounding external environment (thus having a lower chance of contamination).

The systems, methods, and compositions for cryopreservation and reconstitution of engineered tissue compositions is not limited to the engineered tissue compositions herein and may be applied to any other appropriate tissue composition.

Engineered tissue compositions may be cultured over a period of time and then cryopreserved for storage until needed for further processing or end use. Once the tissue composition is needed, it is reconstituted and processed further or used. If processed further, the tissue composition may be cryopreserved again, and later reconstituted. The tissues can be applied using minimally invasive (e.g., percutaneous, catheter, endoscopic, injected, etc.) or surgical (e.g., open, laparoscopic, robotic, etc.) to organs or tissues as a therapeutic. The tissues may also be used as an in vitro testing substrate (e.g., drug toxicology/safety, drug efficacy, drug mechanism of action, disease mechanism of action, etc.).

Tissues may be small (e.g., about 5 mm in diameter) or large (e.g., 5 cm in diameter or greater), or sizes in between. They may contain a single layer of cells, multiple layers of cells, cell nests or aggregates, cell spheroids, or other organizations of cells. The density and organization of the cells may vary depending on the scaffold, extra cellular matrix or similar. The tissue may contain a single type of cell or two types of cells or many types of cells. The cells may be human, or non-human, mammal, or non-mammal. The varying cell populations may be homogeneous, heterogeneous, mixed, or stratified. Tissues may contain a few thousand cells to a few billion cells.

As previously discussed, once tissues complete the desired culture, they may undergo cryopreservation. Tissues may also undergo only one cryopreservation or several intermediate cryopreservations. The purpose of the intermediate cryopreservation may be for storage of an intermediate prior to completion of the process for development of the final tissue product. The purpose of intermediate cryopreservation may also be for activation or encouragement of a specific cellular or tissue process that is cued by temperature changes, or for other purposes.

Cryopreservation can be initiated using a controlled rate freezer or other types or apparatuses such as ethanol jacketed or foam insulated containers that control the rate of freeze. Freezing apparatuses generally have the ability to control changes in temperature at about 1 degree Celsius per minute.

Cryopreservation may be done in vials or in environmental chambers (biobags) varying in shape and volume. Vials are used most often in open system cultures. Environmentally isolated chambers (frequently, but not always called biobags) can be used in an open culture system or a closed culture system. Vials may range, for example, from 0.2 ml to 50 ml or greater in volume. For example, in some embodiments, the cryopreservation is performed in volumes from 0.2-1 ml, 1-5 ml, 5-25 ml (e.g., 10 ml), 10-25 ml, 25 to 50 ml, 50 to 75 ml, 75 to 100 ml, 90 to 110 ml (e.g., 100 ml), etc.

As an example, in some embodiments, a tissue is taken from culture at about 37° C. down to approximately −80° C., in some cases down to about −150° C., and in some cases down to about −196° C.

Cryopreservation is done in the presence of a cryopreservation solution. These solutions may or may not contain dimethyl sulfoxide (DMSO). Those containing DMSO most usually include 2-10% DMSO by volume. They may also contain varying culture mediums such as DMEM, RPMI or similar. They may also contain nutrients for the cells such as FBS or similar. Cryopreservation solutions are well known to one of ordinary skill in the art. Non-limiting examples of commercially available cryopreservation solutions include Cryostor® (BioLife Solutions), CellBanker® (Amsbio), Thermofisher Cryopreservation Medium, etc. The present invention is not limited to any particular cryopreservation solution.

Thawing is the process of taking the tissues from a cryopreserved state (<0° C.) to thawed state, which may be at body temperature (˜37° C.), or room temperature, or a cooled refrigeration (non-frozen) temperature. Thawing may also be considered the process of taking the tissues from the cryopreserved state to a reconstituted state. Reconstitution is the process of taking the tissues from cryopreservation (e.g., a hibernating state) to the full potency required for use. Potency differs based on the tissue and intended use, and may sometimes but not always be related to metabolic activity, gene expression, cytokine expression, viability, etc. Some tissues or applications may require reconstitution to body temperature, whereas others may be reconstituted at a lower temperature or a higher temperature. Further, the reconstitution process may require or allow for intermediate steps at intermediate temperatures where the tissue is either warmed or cooled to temperatures above freezing. The intermediate steps may also be to allow for intermediate term storage until the tissue is needed for use. For example, in some embodiments, the tissue may be thawed to room temperature or wet ice temperature for a period of time (e.g. 24 hours) and later warmed to body temperature. In some embodiments, the tissue may be warmed to body temperature for a period of time and then cooled to room temperature or wet ice temperature for a period of time before use.

Any feature or combination of features described herein are included within the scope of the present invention provided that the features included m any such combination are not mutually inconsistent as will be apparent from the context, this specification, and the knowledge of one of ordinary skill in the art. Additional advantages and aspects of the present invention are apparent in the following detailed description and claims.

### Terms

Unless otherwise explained, all technical and scientific terms used herein have the same meaning as commonly understood by one of ordinary skill in the art to which a disclosed invention belongs. The singular terms “a,” “an,” and “the” include plural referents unless context clearly indicates otherwise. Similarly, the word “or” is intended to include “and” unless the context clearly indicates otherwise. The term “comprising” means that other elements can also be present in addition to the defined elements presented. The use of “comprising” indicates inclusion rather than limitation. Stated another way, the term “comprising” means “including principally, but not necessary solely”. Furthermore, variation of the word “comprising”, such as “comprise” and “comprises”, have correspondingly the same meanings. In one respect, the technology described herein related to the herein described compositions, methods, and respective component(s) thereof, as essential to the invention, yet open to the inclusion of unspecified elements, essential or not (“comprising”).

All embodiments disclosed herein can be combined with other embodiments unless the context clearly dictates otherwise.

Suitable methods and materials for the practice and/or testing of embodiments of the disclosure are described below. Such methods and materials are illustrative only and are not intended to be limiting. Other methods and materials similar or equivalent to those described herein can be used. For example, conventional methods well known in the art to which the disclosure pertains are described in various general and more specific references, including, for example, Sambrook et al., Molecular Cloning: A Laboratory Manual, 2d ed., Cold Spring Harbor Laboratory Press, 1989; Sambrook et al, Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium or Methods from Current Protocols in Molecular Biology, 4th ed., Wiley & Sons, 1999; Harlow and Lane, Antibodies: A Laboratory Manual, Cold Spring Harbor Laboratory Press, 1990; and Harlow and Lane, Using Antibodies: A Laboratory Manual, Cold Spring Harbor Laboratory Press, 1999, Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique; 2^{nd} Ed. (R. I. Freshney, 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.), the disclosures of which are incorporated in their entirety herein by reference.

All publications, patent applications, patents, and other references mentioned herein are incorporated by reference in their entirety for all purposes. In case of conflict, the present specification, including explanations of terms, will control.

Although methods and materials similar or equivalent to those described herein can be used to practice or test the disclosed technology, suitable methods and materials are described below. The materials, methods, and examples are illustrative only and not intended to be limiting, in order to facilitate review of the various embodiments of the disclosure, the following explanations of specific terms are provided:

The term “progenitor cell” refers to cells that have a cellular phenotype that is more primitive (e.g., is at an earlier step along a developmental pathway or progression than is a fully differentiated cell) relative to a cell to which it can give rise to by differentiation. Often, progenitor cells also have significant or very high proliferative potential. Progenitor cells can give rise to multiple distinct differentiated cell types or to a single differentiated cell type, depending on the developmental pathway and on the environment in which the cells develop and differentiate.

The term “stem cell” as used herein, refers to an undifferentiated cell that is capable of proliferation and giving rise to more progenitor cells having the ability to generate a large number of mother cells that can in turn give rise to differentiated, or differentiable daughter cells. The daughter cells themselves can be induced to proliferate and produce progeny that subsequently differentiate into one or more mature cell types, while also retaining one or more cells with parental developmental potential. The term “stem cell” refers to a subset of progenitors that have the capacity or potential, under particular circumstances, to differentiate to a more specialized or differentiated phenotype, and which retains the capacity, under certain circumstances, to proliferate without substantially differentiating. In one embodiment, the term stem cell refers generally to a naturally occurring mother cell whose descendants (progeny) specialize, often in different directions, by differentiation, e.g., by acquiring completely individual characters, as occurs in progressive diversification of embryonic cells and tissues. Cellular differentiation is a process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors. In many biological instances, stem cells are also “multipotent” because they can produce progeny of more than one distinct cell type, but this is not required for “stem-ness,” Self-renewal is the other classical part of the stem cell definition, and it is essential as used in this document. In theory, self-renewal can occur by either of two major mechanisms. Stem cells may divide asymmetrically, with one daughter retaining the stem state and the other daughter expressing some distinct other specific function and phenotype. Alternatively, some of the stem cells in a population can divide symmetrically into two stems, thus maintaining some stem cells in the population as a whole, while other cells in the population give rise to differentiated progeny only.

The term “embryonic stem cell” is used to refer to the pluripotent stem cells of the inner cell mass of the embryonic blastocyst (see U.S. Pat. Nos. 5,843,780, 6,200,806, which are incorporated herein by reference). Such cells can similarly be obtained from the inner cell mass of blastocysts derived from somatic cell nuclear transfer (see, for example, U.S. Pat. Nos. 5,945,577, 5,994,619, 6,235,970, which are incorporated herein by reference). The distinguishing characteristics of an embryonic stem cell define an embryonic stem cell phenotype. Accordingly, a cell has the phenotype of an embryonic stem cell if it possesses one or more of the unique characteristics of an embryonic stem cell such that that cell can be distinguished from other cells. Exemplary distinguishing embryonic stem cell characteristics include, without limitation, gene expression profile, proliferative capacity, differentiation capacity, karyotype, responsiveness to particular culture conditions, and the like.

The term “adult stem cell” or “ASC” is used to refer to any multipotent stem cell derived from non-embryonic tissue, including fetal, juvenile, and adult tissue. Stem cells have been isolated from a wide variety of adult tissues including blood, bone marrow, brain, olfactory epithelium, skin, pancreas, skeletal muscle, and cardiac muscle. Each of these stem cells can be characterized based on gene expression, factor responsiveness, and morphology in culture. As indicated above, stem cells have been found resident in virtually every tissue. Accordingly, the technology described herein appreciates that stem cell populations can be isolated from virtually any animal tissue.

As used herein, the terms “iPS cell” or “induced pluripotent stem cell” refer to a pluripotent cell artificially derived (e.g., induced by complete or partial reversal) from a differentiated somatic cell (from a non-pluripotent cell). A pluripotent cell can differentiate to cells of all three developmental germ layers.

The term “derived from” as applied to a cell being “derived from” another cell or from a tissue means the cell was either isolated from the tissue referred to, or was differentiated from the reference tissue or cell type. Thus, a cell “derived from” a particular individual's tissue was isolated from or differentiated tram that individual's tissue. An individual can include an individual having a given condition. An induced pluripotent stem cell is derived from a somatic tissue of an individual, e.g., a post-partum human individual, frequently an adult. Similarly, and embryonic stem cell is derived from an embryo. A cell derived from an iPS cell refers to a cell that has differentiated from an iPS cell. Alternatively, a cell can be converted from one cell type to a different cell type by a process referred to as transdifferention or direct reprogramming. Alternatively, in the terms of iPS cells, a cell (e.g. an iPS cell) can be derived from a differentiated cell by a process referred to in the art as dedifferentiation or reprogramming.

The term “pluripotent” as used herein refers to a cell that can give rise to any type of cell in the body except germ line cells. The term “pluripotency” or a “pluripotent state” as used herein refers to a cell with the ability to differentiate into all three embryonic germ layers: endoderm (gut tissue), mesoderm (including blood, muscle, and vessels), and ectoderm (such as skin and nerve), and typically has the potential to divide in vitro for a long period of time, e.g., greater than one year or more than 30 passages. Pluripotency is also evidenced by the expression of embryonic stem (ES) cell markers, although the preferred test for pluripotency is the demonstration of the capacity to differentiate into cells of all three germ layers, as detected using, for example, a nude mouse teratoma formation assay. iPS cells are pluripotent cells. Pluripotent cells undergo further differentiation into multipotent cells that are committed to give rise to cells that have a particular function. For example, multipotent cardiovascular stem cells give rise to the cells of the heart, including cardiomyocytes, as well as other cells involved in the vasculature of the heart. Cell useful for in vitro differentiation to myocytes or cardiomyocytes as disclosed herein include, for example, iPS cells as well as multipotent cardiovascular stem cells. A major benefit of the use of iPSC or other stem cells to generate myocytes or cardiomyocytes for the compositions and methods as disclosed herein is the ability to prepare large numbers of such cells and propagate them, e.g., from a specific human patient or subject. This is in contrast to methods, compositions that rely upon the isolation and use of adult cardiac cells.

The term “differentiation” as referred to herein refers to the process whereby a cell moves further down the developmental pathway and begins expressing markers and phenotypic characteristics known to be associated with a cell that are more specialized and closer to becoming terminally differentiated cells. The pathway along which cells progress from a less committed cell to a cell that is increasingly committed to a particular cell type, and eventually to a terminally differentiated cell is referred to as progressive differentiation or progressive commitment. Cell that are more specialized (e.g., have begun to progress along a path of progressive differentiation) but not yet terminally differentiated are referred to as partially differentiated. Differentiation is a developmental process whereby cells assume a more specialized phenotype, e.g., acquire one or more characteristics or functions distinct from other cell types. In some cases, the differentiated phenotype refers to a cell phenotype that is at the mature endpoint in some developmental pathway (a so called terminally differentiated cell). In many, but not all tissues, the process of differentiation is coupled with exit from the cell cycle. In these cases, the terminally differentiated cells lose or greatly restrict their capacity to proliferate. However, in the context of this specification, the terms “differentiation” or “differentiated” refer to cells that are more specialized in their fate or function than at one time in their development. For example in the context of this application, a differentiated cell includes a ventricular cardiomyocyte which has differentiated from cardiovascular progenitor cell, where such cardiovascular progenitor cell can in some instances be derived from the differentiation of an ES cell, or alternatively from the differentiation of an induced pluripotent stem (iPS) cell, or in some embodiments from a human ES cell line. Thus, while such a ventricular cardiomyocyte cell is more specialized than the time in which it had the phenotype of a cardiovascular progenitor cell, it can also be less specialized as compared to when the cell existed as a mature cell from which the iPS cell was derived (e.g. prior to the reprogramming of the cell to form the iPS cell).

A cell that is “differentiated” relative to a progenitor cell has one or more phenotypic differences relative to that progenitor cell and characteristic of a more mature or specialized cell type. Phenotypic differences include, but are not limited to morphologic differences and differences in gene expression and biological activity, including not only the presence or absence of an expressed marker, but also differences in the amount of a marker and differences in the co-expression patterns of a set of markers.

As used herein, “proliferating” and “proliferation” refers to an increase in the number of cells in a population (growth) by means of cell division. Cell proliferation is generally understood to result from the coordinated activation of multiple signal transduction pathways in response to the environment, including growth factors and other mitogens. Cell proliferation may also be promoted by release from the actions of intra- or extracellular signals and mechanisms that block or negatively affect cell proliferation.

The term “tissue” refers to a group or layer of similarly specialized cells that together perform certain special functions.

As used herein, the phrase “cardiovascular condition, disease or disorder” is intended to include all disorders characterized by insufficient, undesired or abnormal cardiac function, e.g., arrhythmia, ischemic heart disease, hypertensive heart disease and pulmonary hypertensive heart disease, valvular disease, congenital heart disease and any condition which leads to congestive heart failure in a subject, particularly a human subject. Insufficient or abnormal cardiac function can be the result of disease, injury and/or aging. By way of background, a response to myocardial injury follows a well-defined path in which some cells die while others enter a state of hibernation where they are not yet dead but are dysfunctional. This is followed by infiltration of inflammatory cells, deposition of collagen as part of scarring, all of which happen in parallel with in-growth of new blood vessels and a degree of continued cell death. As used herein, the term “ischemia” refers to any localized tissue ischemia due to reduction of the inflow of blood. The term “myocardial ischemia” refers to circulatory disturbances caused by coronary atherosclerosis and/or inadequate oxygen supply to the myocardium. For example, an acute myocardial infarction represents an irreversible ischemic insult to myocardial tissue. This insult results in an occlusive (e.g., thrombotic or embolic) event in the coronary circulation and produces an environment in which the myocardial metabolic demands exceed the supply of oxygen to the myocardial tissue.

The term “disease” or “disorder” refers to any alteration in state of the body or of some of the organs, interrupting or disturbing the performance of their functions and/or causing symptoms such as discomfort, dysfunction, distress, or even death to the person afflicted or those in contact with a person. A disease or disorder can also related to a distemper, ailing, ailment, malady, disorder, sickness, illness, complaint, indisposition or affliction.

As used herein, the terms “treat” or “treatment” or “treating” refers to both therapeutic treatment and prophylactic or preventative measures, wherein the object is to prevent or slow the development of the disease, such as slow down the development of a cardiac disorder, or reducing at least one adverse effect or symptom of a cardiovascular condition, disease or disorder, e.g., any disorder characterized by insufficient or undesired cardiac function. Adverse effects or symptoms of cardiac disorders are well-Mown in the art and include, but are not limited to, dyspnea, chest pain, palpitations, dizziness, syncope, edema, cyanosis, pallor, fatigue and death. Treatment is generally “effective” if one or more symptoms or clinical markers are reduced as that term is defined herein. Alternatively, a treatment is “effective” if the progression of a disease is reduced or halted. That is, “treatment” includes not, just the improvement of symptoms or decrease of markers of the disease, but also a cessation or slowing of progress or worsening of a symptom that would be expected in absence of treatment. Beneficial or desired clinical results include, but are not limited to, alleviation of one or more symptom(s), diminishment of extent of disease, stabilized (e.g., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already diagnosed with a cardiac condition, as well as those likely to develop a cardiac condition due to genetic susceptibility or other factors such as weight, diet and health.

The term “scaffold” refers to a support structure for cells and/or cellular material. The support structure may feature fibers and pores, but the scaffold is not limited to such compositions. For example, the scaffold may be in the form of a film, sponge, or solution. The scaffold may be constructed from a variety of materials such as fibers, peptides (e.g., recombinant peptides), lipids, carbohydrates, etc.

### DETAILED DESCRIPTION OF THE INVENTION

The present invention features systems, methods, and compositions for cryopreservation and reconstitution of engineered tissue compositions. Engineered tissue compositions (e.g., three-dimensional tissue compositions that support cell growth and/or maintenance and/or differentiation, etc.) are well known to one of ordinary skill in the art and include but are not limited to the engineered tissue compositions disclosed herein.

For example, the engineered tissue compositions may comprise a scaffold and extracellular matrix (ECM) material. In some embodiments, the ECM may be cell-free, e.g., no cells are present. In some embodiments, the ECM material comprises dead cells, e.g., one living cells. In some embodiments, the ECM material is a hi-product of living cells (currently living cells, dead cells, a combination thereof, etc.), such as but not limited to fibroblasts or any other ECM-generating cell type or a combination thereof. In certain embodiments, the tissue composition comprises a scaffold. ECM material, and a population of cells that are ECM-generating cells, non-ECM-generating cells, or a combination thereof. In certain embodiments, the tissue composition comprises a scaffold, ECM material, and a population of seeded cells (e.g., a cell type of interest). In certain embodiments, the tissue composition comprises a scaffold, ECM material, a population of cells that are ECM-generating cells, non-ECM-generating cells, or a combination thereof, and a population of seeded cells (e.g., a cell type of interest). The aforementioned examples of tissue compositions may comprise additional factors such as growth factors, drugs, compositions for enhancing adherence of cells to the ECM, etc.

### Scaffold

The scaffold used for the engineered tissue compositions herein may be constructed from a variety of types of materials, a variety of sizes of materials, a variety of configurations, etc. In certain embodiments, the scaffold comprises a plurality of fibers, wherein the fibers are arranged (e.g., woven) together yielding a plurality of pores disposed therein between. The scaffold is not limited to a fiber configuration. In certain embodiments, the scaffold comprises a film, a sponge, a gel, a solution, etc. A non-limiting example of an alternative scaffold is a sponge or film constructed from polymers, proteins, recombinant peptides, e.g., Human Collagen Type I. Sponges and films are further described below.

For embodiments with scaffolds in a fiber configuration, the fibers of the scaffold may be generally uniform in diameter or the fibers may be of various diameters. For example, in some embodiments, the scaffold comprises fibers of a first fiber type and fibers of a second fiber type, wherein the first fiber type has a diameter different from that of the second fiber type. Note the scaffold may comprise more than a first fiber type and a second fiber type, e.g., the scaffold may comprise a first fiber type and second fiber type and a third fiber type, or further a fourth fiber type, or further a fifth fiber type, etc. Scaffolds with more than one fiber type may be arranged in a variety of configurations including but not limited to a printed or spun fiber or knit or weave that has a fixed pattern of fiber type arrangement, a printed or spun fiber or knit or weave that has a random fiber type arrangement, etc. in some embodiments, small fibers extend from one or more large fibers. In some embodiments, the fibers are loosely printed or spun or knitted or woven such that space exists for cells to position therein between. The fibers may be arranged in a configuration and/or orientation that allows for a particular cell alignment.

As previously discussed, the fibers of the scaffold may be arranged in a weave or knit configuration. Weave or knit configurations may include but are not limited to a plain weave, a twilled weave, an alternating twilled weave, a knitted weave a plain Dutch weave, a Dutch twilled weave, a reverse Dutch weave, a hexagonal open worked stitch weave, a warp knit, or a combination thereof. For construction purposes, the scaffold may be weaved, knit, spun, extruded, or printed. In some embodiments, the scaffold has a ring-like configuration.

Fiber diameters may be of various sizes. For example, in some embodiments, at least a portion of the fibers have a diameter that is from 5 μm to 100 μm, from 10 μm to 500 μm, from 100 μm to 1 mm (e.g., fiber bundles), etc. The present invention is not limited to the aforementioned fiber diameters.

In some embodiments, the fibers are all constructed from a single material. In some embodiments, a portion of the fibers is constructed from a first material and a portion of the fibers is constructed from a second material different from the first material. In some embodiments, a portion of the fibers is constructed from a first material, a portion of the fibers is constructed from a second material different from the first material, and a portion of the fibers is constructed from a third material different from the first and second materials. The present invention is not limited to three different material types; the scaffold may be constructed from four different materials, five, six, etc. In some embodiments, one or more fibers of the scaffold are constructed from two or more materials, e.g., individual fibers are made from a combination of materials.

Materials used for constructing the scaffold (e.g., a scaffold with fibers and/or other components such as peptides) may include but are not limited to polyglycolide, polylactide, polyhydroxobutyrate, poly(anhydrides), poly(dioxanone), poly (trimethylene carbonate), polyglactin, poly(lactic acid), polyvinylidene fluoride, polyesters, silicone, polyurethane, polymethylmethacrylate, polypropylene, polyethylene, poliglecaprone-25 monofilament, a polycarbonate, a polyamide, a polyesters, a polystyrene, a polyacrylate, a polyvinyl, polytetrafluorethylene, thermanox, nitrocellulose, collagen, elastin, silk, metals, TMC, polyester, gelatin, dextran, proteins, peptides, or a combination thereof. For example, in some embodiments, the test material comprises at least glycolide, lactate and trimethylene carbonate, and the second material comprises at least lactide and trimethylene carbonate.

As previously discussed, in certain embodiments, the scaffold is in the form of a film. In certain embodiments, the scaffold is in solution. In certain embodiments, the scaffold is a sponge. Regarding the sponge configuration, in some embodiments, the sponge has a generally uniform diameter. In certain embodiments, the sponge has a non-uniform diameter. In certain embodiments, the sponge is stratified having multiple levels or layers (e.g., a lower level, an upper level, etc.), wherein one layer may have a first uniformity and a different layer may have a second uniformity. The sponge may feature grooves or ridges. For example, one layer, e.g., a top level or a bottom layer, may feature ridges or grooves.

The scaffold may be constructed from a variety of materials including but not limited to peptides (e.g., recombinant peptides), carbohydrates, lipids, etc. In certain embodiments, the scaffold comprises both recombinant peptides and fibers (e.g., absorbable/degradable fibers, non-absorbable/degradable fibers, or a combination thereof). A non-limiting example of a recombinant peptide used for creating a film scaffold or sponge scaffold or solution scaffold includes collagen type I.

The scaffold may be constructed from biologically-derived material, synthetically-derived material, or a combination of synthetically-derived and biologically-derived material.

The scaffold may feature grooves and ridges, e.g., parallel grooves and ridges, organized or patterned grooves and ridges, randomly organized grooves and ridges. The configuration of the scaffold may help enhance proliferation or differentiation/maturation of a cell. The configuration of the scaffold may help organize the cells in a particular direction or orientation, e.g., align the cells for a particular purpose such as muscle contraction.

The scaffold may be constructed in a variety of thicknesses. For example, in some embodiments, the scaffold is at least 10 um thick. In some embodiments, the scaffold is at least 25 um thick. In some embodiments, the scaffold is at least 40 um thick. In some embodiments, the scaffold is at least 50 um thick. In some embodiments, the scaffold is at least 100 um thick. In some embodiments, the scaffold is at least 250 um thick. In some embodiments, the scaffold is at least 500 um thick. In some embodiments, the scaffold is at least 1 mm thick. In some embodiments, the scaffold is at least 2 mm thick. In some embodiments, the scaffold is from 30 or 40 um to 820 or 850 um. In some embodiments, the scaffold is from 50 um to 500 um thick. In some embodiments, the scaffold is from 500 um to 1 mm thick. In some embodiments, the scaffold is from 1 mm to 2 mm thick. The present invention is not limited to the aforementioned thicknesses, e.g., 2 to 3 mm, 3 to 4 mm, 4 to 5 mm, 5 to 6 mm, etc. For example, the thickness may be from 45 um to 1070 um, 45 to 499 um, 246 to 1070 um, etc. The aforementioned thicknesses may apply to the engineered tissue composition (e.g., the scaffold with ECM optionally with cells, etc.). For example, in certain embodiments, the engineered tissue composition is from 200-1000 microns in thickness.

In some embodiments, at least a portion of the tissue composition is absorbable. Some components (or all of the components) of the scaffold, e.g., fibers, peptides, etc. may be resorbable, absorbable, or degradable. For example, one or more of the components may resorb/absorb/degrade/dissolve as proliferative ECM-generating cells replicate on and in the scaffold. In an example wherein the scaffold comprises two or more different fiber types, in certain embodiments, fibers of a first fiber type may be resorbable/absorbable/degradable and fibers of a second fiber type may not be resorbable/absorbable/degradable. In certain embodiments, all of the scaffold components may be resorbable/absorbable/degradable in some capacity, e.g., a first fiber type may be resorbable/absorbable/degradable at a rate that is different than that of a second fiber type. The scaffold may feature a degradation profile (timed, tiered), wherein portions of the scaffold degrade at particular times. Any appropriate degradable materials or combinations thereof may achieve a desired degradation profile. For the examples below, the degradation profile (when the scaffold resorbs/absorbs/degrades) is measured starting from the time of surgical implantation of the tissue composition. In some embodiments, a portion or all of the scaffold resorts/absorbs/degrades within 1 day, within 2 days, within 3 days, within 4 days, within 5 days, within 6 days, within 1 week, within 8 days, within 9 days, within 10 days, within 11 days, within 12 days, within 13 days, within 2 weeks, within 3 weeks, within 1 month, within 2 months, within 3 months, within 4 months, within 5 months, within 6 months, within 1 year, within 2 years, within 3 years, within 4 years, within 5 years, etc. In certain embodiments the scaffold does not fully resorb/absorb/degrade (the scaffold is non-absorbable).

The mechanical properties (e.g., stiffness, etc.) of the scaffold may change as components (e.g., fibers and/or other materials such as peptides) of the scaffold resorb/absorb/degrade.

Without wishing to limit the present invention to any theory or mechanism, it is believed that a scaffold that does not significantly curl up or fold over on itself during surgical implantation may provide advantages, e.g., the tissue composition may be easier for the surgeons to implant if it does not fold over on itself.

In certain embodiments the scaffold comprises absorbable/degradable fibers, non-absorbable/degradable fibers, or a combination thereof. In certain embodiments, the scaffold comprises a sponge (e.g., constructed with recombinant peptides, e.g., Human Collagen Type I). In some embodiments, the scaffold comprises a film (e.g., constructed with recombinant peptides, e.g., Human Collagen Type I). In some embodiments, the scaffold is in solution. In certain embodiments, the scaffold comprises both recombinant peptides (forming a film or sponge) and fibers (e.g., absorbable/degradable fibers, non-absorbable/degradable fibers, or a combination thereof). In certain embodiments, the scaffold comprises biologically-derived components (e.g., naturally produced by cells), synthetically-derived components, or a combination thereof. In certain embodiments, the scaffold comprises additional features or properties that enhances the adherence of cells and/or ECM. For example, in certain embodiments, the scaffold has a hydrophilicity adapted to allow adherence of cells and/or ECM. In certain embodiments, the scaffold has a surface roughness adapted to allow adherence of cells and/or ECM. Non-limiting examples of compositions or features that may enhance cell adherence may include certain textures or roughness, certain hydrophilic compounds or features, certain ligands, RGD-coated materials, etc.

The scaffold may be anisotropic, the mechanical properties of the scaffold may be different in one direction than the other. Or, the scaffold may be isotropic. The scaffold has a variety of mechanical properties related to strength and flexibility.

There is generally a difference between the base material properties (e.g., the scaffold alone) and the properties of the tissue composition (with the fibroblasts and ECM). For example, if the scaffold features components (e.g., fibers and/or peptides) that are degradable (e.g., fibers or peptides that degrade during culture), the scaffold may be stiffer than what is ultimately used as the tissue composition (e.g., for in vivo use). In some embodiments, a scaffold may be chosen with a material in the GPa range, but when the tissue composition is implanted, it may be in the MPa range, likewise, a scaffold may be chosen in the MPa range but when the tissue composition is implanted it may be in the kPa range. In some embodiments, the implanted materials (the property of the tissue composition) may be below 100 MPa.

The elastic modulus (stiffness) may be from 20 kPa to 100 GPa. The scaffold may have a burst strength from 20 N/cm to 200 N/cm, from 50 N/cm to 100 N/cm, from 75 N/cm to 90 N/cm, etc. The scaffold may have a parallel/perpendicular tear resistance from 10N/5N to 50N/40N. The scaffold may have a parallel/perpendicular tear resistance from 30/31N to 350N/36N. In some embodiments, the scaffold has a longitudinal stiffness from 0.1 N/mm to 50 N/mm. In some embodiments, the scaffold has a longitudinal stiffness from 1 N/mm to 30 N/mm. In some embodiments, the scaffold has a transverse stiffness from 0.5 N/mm to 5 N/mm. In some embodiments, the scaffold has a longitudinal stiffness that is different from a transverse stiffness. In some embodiments, the scaffold has a longitudinal stiffness that is the same as a transverse stiffness. In some embodiments, the scaffold has a longitudinal maximum force from 1 kPa to 100 MPa. In some embodiments, the scaffold has a transverse maximum force from 1 kPa to 100 MPa. In some embodiments, the scaffold has a strength from 1 to 3000 kPa. In some embodiments, the scaffold has a strength from 3000 to 4600 kPa. In some embodiments, the scaffold has a strength greater than 4600 kPa. The scaffold may feature fibers with different stiffness e.g., fibers with stiffness of 0.1-20 N/mm and fibers with strength >10 Mpa. As previously discussed, the stiffness of the scaffold prior to culturing of cells may be different from the end product, e.g., the tissue composition. In some embodiments, the tissue composition strength (e.g., end product) may be from 1 kPa to 50 MPa, however the present invention is not limited to those values.

The mechanical properties (e.g., stiffness, etc.) may change over the course of manufacturing, cell proliferation, cell differentiation, implantation, etc. The present invention is not limited to the mechanical property parameters described herein.

In certain embodiments, the engineered tissue composition allows for electrical signal transduction.

The density of pores, e.g., number of pores per unit of area (e.g., number of pores per mm² of scaffold), may help cells to efficiently grow across the scaffold. In some embodiments, the pores are closely spaced and/or pores are positioned above pores slightly overlapping; however, the present invention is not limited to closely spaced pores or pores positioned above pores slightly overlapping. Further, the density of pores may depend on the type of material used for the scaffold, the thickness of the scaffold, the type of weave of the scaffold, etc. In some embodiments, the scaffold has from 1 to 1,000 pores per cm². In some embodiments, the scaffold has from 10 to 1,000 pores per cm². In some embodiments, the scaffold has from 100 to 1,000 pores per cm². In some embodiments, the scaffold has from 100 to 1,000 pores per mm². In some embodiments, the scaffold has from 100 to 500 pores per mm². In some embodiments, the scaffold has from 200 to 1,000 pores per mm². The pore density may also change over time. For example, in some embodiments, components of the scaffold (e.g., fibers and/or peptides, etc.) may degrade (e.g., biodegrade, resorb, absorb, etc.), yielding a different pore density than what was originally present in the scaffold. In some embodiments, the pores are arranged uniformly throughout the scaffold. In some embodiments, the pores are arranged randomly throughout the scaffold. In some embodiments, the pores are arranged in a pattern throughout the scaffold. The pores may be of various shapes (e.g., cross-sectional shapes), e.g., rectangular, rounded rectangular, or of other geometric shape or irregular shape or shape combination including but not limited to hexagonal, circular, oval, figure eight-shaped, etc. Thus, the pores may be described as having a height, width, length, diameter, area, etc. In some embodiments, the pores (e.g., one or more of the pores) of the scaffold have an area from 0.1 μm² to 100 μm², from 1 μm² to 1000 μm², from 100 μm² to 5000 μm², from 0.1 μm² to 0.01 mm², from 0.1 μm² to 0.1 mm², from 0.1 μm² to 1 mm², from 0.1 μm² to 2 mm², from 0.1 μm² to 10 mm², etc. In some embodiments, the average pores size is approximately 104,540 μm². In some embodiments, the average pore size is from 5,000 μm² to over 1,000,000 μm². In some embodiments, the pores have a diameter that is from 50 μm to 90 μm. In some embodiments, the pores have a diameter from 50 μm to 200 μm. In some embodiments, the pores have a diameter from 200 μm to 400 μm, 200 μm to 500 μm, etc. In some embodiments, the pores have a diameter from 500 μm to 1000 μm. In some embodiments, the pores have a diameter from 500 μm to 1500 μm. In some embodiments, the pores have a diameter from 800 μm to 1200 μm. In some embodiments, the pores have a diameter from 800 μm to 1000 μm. In certain embodiments, the pore size is from 500 μm to 1200 μm.

In some embodiments, the scaffold retains at least 50% of its mechanical strength for at least 4 weeks, at least 5 weeks, at least 8 weeks, at least 10 weeks, at least 15 weeks, at least 20 weeks, at least 30 weeks, at least 40 weeks, etc. In some embodiments, the scaffold degrades in no less than 3 weeks. In some embodiments, the scaffold degrades in no less than 4 weeks after implantation. In some embodiments, the scaffold degrades in no less than 6 weeks after implantation. In some embodiments, the scaffold degrades in no less than 8 weeks after implantation. In some embodiments, the scaffold degrades in no less than 10 weeks after implantation. The scaffold may feature two different fibers, wherein one is fast resorbing and one is slow resorbing (relative to each other), to allow for dual-stage resorption.

### Extracellular Matrix Material

As previously discussed, the engineered tissue compositions of the present invention comprise extracellular matrix (ECM) material. The ECM may be produced by ECM-generating cells, however the present invention is not limited to ECM produced by ECM-generating cells. In certain embodiments, the ECM comprises only synthetically-derived material. In certain embodiments, the ECM comprises biologically-derived material (e.g., ECM produced by ECM-generating cells). In certain embodiments, the ECM comprises a combination of biologically-derived material and synthetically-derived material. As an example, materials such as synthetically-produced collagen and fibronectin (and the like, e.g., materials discussed herein) may be combined to form an ECM without the need for ECM-generating cells. In some embodiments, from 0 to 10% of the ECM or tissue composition (by area or volume) is synthetically-derived. In some embodiments, from 10 to 25% of the ECM or tissue composition (by area or volume) is synthetically-derived. In some embodiments, from 25 to 40% of the ECM or tissue composition (by area or volume) is synthetically-derived. In some embodiments, from 40 to 60% of the ECM or tissue composition (by area or volume) is synthetically-derived. In some embodiments, from 60 to 75% of the ECM or tissue composition (by area or volume) is synthetically-derived. In some embodiments, from 75 to 90% of the ECM or tissue composition (by area or volume) is synthetically-derived. In some embodiments, from 50 to 95% of the ECM or tissue composition (by area or volume) is synthetically-derived.

Components of the ECM may include but are not limited to collagen (e.g., collagen type I, collagen Type III), elastin, fibronectin, laminins, tenascin, proteoglycans, glycosaminoglycans (e.g., Verlscan, Decorin, Betaglycan, Syndecan), etc. (see Naughton, 2002. Ann N Y Acad Sci. 961:372-85). In some embodiments, exogenous gelatin is deposited on the scaffold. In some embodiments, exogenous collagen, fibronectin, fibrin is added (or other appropriate ECM components).

Without wishing to limit the present invention to any theory or mechanism, it is believed that a certain amount of ECM is beneficial for the engineered tissue composition to be effective (e.g., effective for accepting seed cells, for differentiating seed cells, for surgical implantation, etc.).

In certain embodiments, the ECM is produced by seeding ECM-generating cells in and/or on the scaffold (e.g., on and/or within the pores and components of the scaffold), wherein the ECM-generating cells subsequently proliferate and expand in and/or on and through the scaffold, producing ECM. The ECM-generating cells may migrate along the components (e.g., fibers, peptides, etc.) of the scaffold and further within the pores (e.g., along with ECM that is generated). The seeding process for the ECM-generating cells (and/or otter cells herein) may utilize various steps to enhance adherence of the cells onto the scaffold, such as but not limited to centrifugation or other appropriate forces (e.g., electrical force), or combinations thereof. In certain embodiments, the ECM is produced by ECM-generating cells prior to the application of the ECM onto the scaffold. As an example, following production of the ECM by the ECM-generating cells, the ECM material along with the ECM-generating cells may be applied to the scaffold. Alternatively, in certain embodiments, the ECM material produced by the ECM-generating cells may be made cell-free and subsequently applied to the scaffold. In certain embodiments, a population of cells is seeded in and/or on the scaffold prior to the application of the ECM.

Note that the ECM-generating cells may be live or dead (or a combination of live and dead cells). For example, the tissue composition may comprise the scaffold, ECM, and live ECM-generating cells (e.g., fibroblasts or other ECM-generating cell type or a combination thereof). In certain embodiments, the tissue composition comprises the scaffold, ECM, and dead ECM-generating cells. In certain embodiments, the tissue composition comprises the scaffold, ECM, and a population of live ECM-generating cells and a population of dead ECM-generating cells.

The ECM-generating cells may be fibroblasts, e.g., human dermal fibroblasts. However, the present invention is not limited to fibroblasts. In some embodiments, the ECM-generating cells comprise fibroblasts, osteoblasts, chondrocytes, glial cells, neural stem cells, cardiomyocytes, myofibroblasts, the like, or a combination thereof. As discussed herein, in certain embodiments, the ECM-generating cells may be genetically engineered to produce specific ECM and/or growth factors and/or engineered to proliferate, etc.

The ECM-generating cells may be derived from an appropriate source or host. For example, in some embodiments, the ECM-generating cells are human cells. In some embodiments, the ECM-generating cells are primate cells. In some embodiments, the ECM-generating cells are mouse cells, rat cells, goat cells, rabbit cells, horse cells, canine, feline, or any other host-derived cells. In certain embodiments, the ECM-generating cells are genetically modified to be universal cells (non-immunogenic).

As previously discussed, the ECM-generating cells may be fibroblasts. In certain embodiments, the fibroblasts are iPSC-derived fibroblasts. In some embodiments, the fibroblasts are skin-derived fibroblasts, e.g., dermal neonatal fibroblasts. In some embodiments, the fibroblasts are blood-derived fibroblasts. In some embodiments, the fibroblasts are heart-derived, muscle-derived, liver-derived, pancreas-derived, adipose tissue-derived, central nervous system (CNS)-derived, or lung-derived fibroblasts.

In certain embodiments, the ECM-generating cells are wild type cells. In certain embodiments, the ECM-generating cells are genetically modified, e.g., engineered to express one or more genes of interest. In certain embodiments, the ECM-generating cells are a combination of wild type and genetically modified cells.

The ECM-generating cells may form a layer atop the scaffold. (In certain embodiments, the ECM cells that are seeded on/in the scaffold are already in ECM. In certain embodiments, cells in the ECM are alive and/or dead.) in some embodiments, the ECM-generating cells are disposed within and/or on top of the scaffold. The ECM-generating cells may be present in aggregates in or on top of the scaffold, form one or more layers on the scaffold, adopt an alternative arrangement within or on top of the scaffold, or a combination thereof. The tissue compositions of the present invention may have layers of cells, e.g., from 3 to 500 cell layers. The cell layers may be made up of ECM-generating cells, non-ECM generating cells, or a combination thereof.

As ECM-generating cells proliferate and produce ECM in and on the scaffold, the ECM-generating cells and/or the ECM fill in at least a portion of the pores of the scaffold. The ECM-generating cells and/or the ECM may then fill in all of the pores of the scaffold. Note in some embodiments, a cell free ECM material is used that fills in a portion or all of the area of the pores of the scaffold.

With respect to tissue compositions comprising scaffold made from recombinant peptides, in certain embodiments, the recombinant peptide scaffold is from 300 to 500 μm thick. In certain embodiments, the cardiomyocyte later is from 20 to 50 μm thick. In certain embodiments, the pores are from 50 to 90 μm in diameter. The present invention is not limited to the aforementioned dimensions.

In some embodiments, 100% of the area of the pores is filled by the ECM-generating cells and/or the ECM. In some embodiments, at least 99%, at least 98%, at least 97%, at least 96%, at least 95%, at least 90%, at least 85%, at least 80%, at least 75%, at least 70%, at least 50%, etc. of the area of the pores is filled by the ECM-generating cell and/or the ECM.

In some embodiments, at least 50% of the area of the pores is filled (filled by the ECM-generating cells and/or the ECM) within 2 to 10 days of seeding the ECM-generating cells, within 3 to 10 days of seeding the ECM-generating cells, within 4 to 10 days of seeding the ECM-generating cells, within 5 to 10 days of seeding the ECM-generating cells, within 8 to 10 days of seeding the ECM-generating cells, within 5 to 15 days of seeding the ECM-generating cells, within 8 to 15 days of seeding the ECM-generating cells, within 10 to 15 days of seeding the ECM-generating cells, within 12 to 15 days of seeding the ECM-generating cells, within 5 to 25 days of seeding the ECM-generating cells, within 10 to 25 days of seeding the ECM-generating cells, within 15 to 25 days of seeding the ECM-generating cells, within 20 to 25 days of seeding the ECM-generating cells, etc.

The time it takes for the pores to fill may depend on certain factors, e.g., how the cells are seeded, e.g., whether or not the tissue composition is rocked during the seeding process, rocking rates, whether centrifugation was used during the seeding process, etc. As an example, with rocking, in certain embodiments, at least 50% of the area of the pores is filled (filled by the ECM-generating cells and/or the ECM) within 5 to 10 days of seeding the ECM-generating cells, whereas in certain embodiments without rocking, at least 50% of the area of the pores is filled (filled by the ECM-generating cells and/or the ECM) within 14-17 days of seeding the ECM-generating cells. The aforementioned example is not mean to limit the present invention in any way and merely serves as an example to describe that rocking may accelerate the time needed to fill at least 50% of the area of the pores.

As the ECM-generating cells proliferate and generate ECM in the scaffold, the cells undergo morphological changes. For example, after a certain number of days following seeding with human neonatal dermal fibroblasts (HDF), there is a longer spindle shaped morphology of the HDF. Later, there is a more homogenous population of cells that do not display the spindle shaped morphology but instead a small pebbled morphology. In some embodiments, the long spindle shaped morphology may form at a time less than 17 days, e.g., in 16 days, 15 days, 14 days, 13 days, 12 days, 11 days, 10 days, 9 days, 8 days, 7 days, 6 days, 5 days, 4 days, less than 4 days, 16 days or less, 15 days or less, 14 days or less, 13 days or less, 12 days or less, 11 days or less, 10 days or less, 9 days or less, 8 days or less, 7 days or less, 6 days or less, 5 days or less, etc. In certain embodiments, the small pebbled morphology may form at a time less than 28 days, e.g., in 27 days, 26 days, 25 days, 24 days, 23 days, 22 days, 21 days, 20 days, 19 days, 18 days, 17 days, 16 days, 15 days, 14 days, 13 days, 12 days, 11 days, 10 days, 9 days, 8 days, less than 8 days, 26 days or less, 25 days or less, 24 days or less, 23 days or less, 22 days or less, 21 days or less, 20 days or less, 19 days or less, 18 days or less, 17 days or less, 16 days or less, 15 days or less, 14 days or less, 13 days or less, 12 days or less, 11 days or less, 10 days or less, 9 days or less, 8 days or less, etc. The time it takes for the morphological changes to occur can depend on the manufacturing method.

The engineered tissue compositions may comprise a final density of ECM-generating cells from 5×10⁵ cells/cm² to 5×10⁸ cells/cm². In some embodiments, the engineered tissue compositions comprise a final density of ECM-generating cells from 1×10 cells/cm² to 1×10⁷ cells/cm². In some embodiments, the engineered tissue compositions comprise a final density of ECM-generating cells from 1×10⁴ cells/cm² to cells/cm². The present invention is not limited to the aforementioned final densities of ECM-generating cells.

In some embodiments, additional factors are added to the ECM, the scaffold, and/or the ECM-producing cells. Additional factors may be added to enhance the production of ECM, or for other purposes such as for enhancing cell growth, maintenance, and/or differentiation, for enhancing adherence of cells and/or ECM, etc. As a non-limiting example, ascorbic acid, which helps drive ECM deposits, may be added. In some embodiments, the additional factor is for enhancing adherence of the ECM-generating cells and/or for enhancing adherence of seeded cells.

In some embodiments, exogenous growth factors are added either along or in combination with the ECM and/or ECM-generating cells and/or the scaffold. The growth factors (e.g., those secreted by the fibroblasts, those added exogenously) may improve proliferation (of the fibroblasts themselves, of the seeded cells), seeding efficiency (of the fibroblasts themselves, of the seeded cells), integration of the fibroblasts into the scaffold, generation of the ECM, etc. Growth factors may include but are not limited to vascular endothelial growth factor (VEGF), fibroblast growth factors (e.g., basic fibroblast growth factor (bFGF)), hepatocyte growth factor (HGF), angiopoietin-1, matrix deposit factors (e.g., Transforming growth factor (TGF-b, TGF-b1), Transforming growth factor (TBG-b3)), mitogenic factors (e.g., Platelet derived growth factor A (PDGF-A), Insulin like growth factor 1 (IGF-1), Erythropoietin (EPO), Heparin binding epidermal growth factor (HBEGF). Transforming growth factor a (TGFa)), angiogenic factors (e.g., Angiogenein, Angiopoientin-2), Endothelial Growth Factor, Leptin, Platelet derived growth factor BB (PDGF-BB), Secreted protein acid and rich in cysteine (SPARC), Interleukin 6 (IL-6), Interleukin 8 (IL-8), Inflammatory Cytokines (e.g., Interferon-gamma, Interleukin 1a, Interleukin 1b, Interleukin 6 (IL-6), Interleukin 8 (IL-8), Monocyte chemotactic protein 1, Granulocyte colony stimulating factor (GCSF), Tumor necrosis factor a (TNFa)), etc. (see Naughton, 2002, Ann N Y Aced Sol, 961:372-85; Lancaster et al, 2010, Tissue Eng Part A, 16(10):3065-73). Growth factors from other cells, e.g., therapeutic cells, may be added. For example, cardiomyocytes secrete specific factors to stimulate in vitro signaling, maturation, myokine activities and in viva myogenesis, etc., and said growth factors may also be added exogenously or via seeding of another cell type.

In some embodiments, the scaffold makes up between 1 to 70% of the tissue composition by volume. In some embodiments, the scaffold makes up between 1 to 80% of the tissue composition by volume. In some embodiments, the scaffold makes up between 5 to 50% of the tissue composition by volume. In some embodiments, the ECM and ECM-generating cells make up at least 20% of the tissue composition by volume. In some embodiments, the ECM and ECM-generating cells make up from 20 to 50% of the tissue composition by volume. In some embodiments, the ECM and ECM-generating cells make up from 50 to 75% of the tissue composition by volume. In some embodiments, the ECM and ECM-generating cells make up from 50 to 99% of the tissue composition by volume. In some embodiments, the ECM and ECM-generating cells make up from 30 to 99% of the tissue composition by volume. The amount of the tissue composition made up of ECM and ECM-generating cells (vs. scaffold) may depend on various factors, e.g., amount of ECM-generating cells seeded at the start of culture, the expected degradation rate of scaffold material, the amount of seed cell population, etc. For example, the amount of the tissue composition made up of seed cells may range from 3 to 60% of the tissue composition.

As an example, specific tissue compositions (comprising ECM and ECM generating cells) constructed with particular scaffold materials (e.g., a dual-fiber/slower degrading scaffold, a lactide scaffold, a polyglactin scaffold) were analyzed for assessing the mass of the cells and ECM relative to scaffold mass. First the scaffold was weighed, and then the tissue composition (fully cultured HDF-scaffold composition) was weighed both wet and dry. With the dual-fiber scaffold cultured with cells and ECM, the wet weight was 0.058 g/cm2 with a dry weight of 0.028 g/cm2 whereas the dual-fiber scaffold alone had a wet weight of 0.021 g/cm2 and dry weight of 0.016 g/cm2. Thus the mass of ECM and ECM depositing cells was 0.037 g/cm2, The dry weight was 0.012 g/cm2.

The engineered tissue composition may be generally flat. The engineered tissue composition may itself have a curl, or microscopic features of the tissue composition may have convex or concave components. Without wishing to limit the present invention to any theory or mechanism, it is possible that a concave structure may be beneficial for seeding, adhesion, and integration of cells.

As previously discussed, in certain embodiments the ECM is biologically-derived (e.g., from ECM-generating cells), synthetically-derived, or a combination thereof. In certain embodiments, the ECM-generating cells are wild type, genetically modified, or the ECM-generating cells features a population of wild type cells and a population of genetically modified cells. In certain embodiments, the ECM is cell free. In certain embodiments, the ECM comprises ECM-generating cells that are live, dead, or feature a population of live cells and a population of dead cells. In certain embodiments, the ECM comprises growth factors, drugs, and/or other compositions that help ECM production, cell adherence, ECM adherence to the scaffold, etc.

### Seeded Cells

The tissue compositions of the present invention may comprise seeded cells. The seeded cells may be or any appropriate cell type (and from any appropriate host or genetically modified to be universal cells). For example, in some embodiments, the seed cells are human cells. In some embodiments, the seed cells are mouse cells, rat cells, goat cells, rabbit cells, horse cells, canine, feline, or any other host-derived cells. The seed cells may be associated with blood, cardiac tissue, skeletal muscle tissue, liver tissue, pancreatic tissue, lung tissue, bone tissue, umbilical cord tissue, endothelial tissue, central nervous system tissue, gastrointestinal tissue, endocrine cells, paracrine cells, enzyme-secreting cells, stem cells thereof, progenitors thereof, prokaryote, eukaryote or other oxygen emitting particle, or a combination thereof. Note the seeded cells may be from the same donor as the ECM-generating cells. In certain embodiments, the seeded cells are from a donor different from that of the ECM-generating cells.

The seeded cells may be proliferative, non-proliferative, or a combination thereof. The seeded cells may be stem cells (e.g., adult stem cells, embryonic stem cells, induced pluripotent stem cells), primary cells, progenitor cells, etc. For example, the seeded cells may be human inducible pluripotent stem cell-derived cells (hiPSCs), e.g., human inducible pluripotent stem cell-derived cardiomyocytes (hiPSC-CMs) The seed cells may be terminally differentiated cells, e.g., terminally differentiated cardiomyocytes, hepatocytes, beta cells, endoderm, smooth muscle cells, salivary cells, etc.

The seeded cells may be mature or immature. For example, cardiac progenitor cells may express one or more markers such as but not limited to MESP1, GATA4, ISL1, NKX2.5, the like, or a combination thereof. Nascent cardiomyocytes may express one or more markers such as but not limited to CTNT, MHC, MLC, sarcomeric actinin, the like, or a combination thereof. iPSCs may express one or more markers such as but not limited to Oct-4, LIN-23, the like, or a combination thereof.

The seeded cells may be wild type cells. In certain embodiments, the seeded cells are genetically modified to express one or more genes of interest. In certain embodiments, the seeded cells comprise a population of wild type cells and a population of genetically modified cells. For example, genes of interest may include but are not limited to thymosin beta-4 (TB4), akt murine thyoma viral oncogene homolog (AKT1), stroma cell-derived factor-1 alpha (SDF-1), hepatocyte growth factor (HGF), insulin like growth factor one (IGF-1), erythropoietin (EPO), etc. The present invention is not limited to the aforementioned genes, nor is the present invention limited to genetically modified cells that express a gene for a particular therapeutic purpose. In some embodiments, included with the seeded cells may be additional cells or particles such as prokaryotes, eukaryotes, or particles engineered to produce oxygen spontaneously or with external stimulation.

Cells from a particular disease state or genetic condition may be seeded. For example, the seed cells may be cells having an abnormality associated with a particular disease state or condition. In some embodiments the seed cells are cells derived from a tissue in an abnormal state (e.g., subsequent to a stress or trauma or event such as a myocardial infarction). Non-limiting examples of cells with particular genetic mutations or cells associated with a particular disease state or condition may include those associated with congenital cardiomyopathies, acquired cardiomyopathies, arrythmogenic cardiomyopathies (e.g., long QT syndrome, short QT syndrome (SOTS), Brugada syndrome, catecholaminergic polymorphic ventricular tachycardia (CPVT), arrythmogenic right ventricular cardiomyopathy (ARVC)), dilated cardiomyopathies (e.g., hypertrophic cardiomyopathy, left ventricular noncompaction, transthyretin amyloidosis, hereditary hemochromatosis, RASopathis (also known as Noonan spectrum disorders), heart failure, etc. In some embodiments, the disease state or condition is an acquired condition such as dilated ischemic and on-ischemic cardiomyopathies, hypertensive heart disease, etc. In some embodiments, the disease state or condition is a congenital disease acquired or congenital plus arrhythmia. In some embodiments, the disease state or condition is diabetes, a cancer, muscular dystrophy, a congenital, genetic, or acquired condition affecting the GI tract, a condition affecting skeletal muscle, smooth muscle, etc. The present invention is not limited to the aforementioned conditions.

The ECM-generating cells and/or the ECM and/or other factors of the tissue compositions may cause the differentiation and/or maturation of the seeded cells (if appropriate). Growth factors (e.g., fibroblast-derived, exogenous) may help improve one or more of: seeding, integration into the scaffold, proliferation, and differentiation of the seeded cells in vitro or in vivo. For certain cells with the ability to differentiate into two or more cell types, the microenvironment of the tissue composition (e.g., ECM-generating cells, ECM, growth factors, etc.) can help drive the pathway of differentiation. In some embodiments, exogenous factors are added to enhance differentiation in a particular direction.

The tissue compositions of the present invention may further comprise enhancement cells. Non-limiting examples of enhancement cells may include secretory cells, paracrine cells, enzymatic cells, beta cells, gastrointestinal cells, or a combination thereof. Enhancement cells may be at a particular ratio with respect to the seeded cells and/or ECM-generating cells. The ratio of the cells may depend on the cell type and a desired outcome. The ratio may also depend on clustering of the enhancement cells (e.g., cell bundles). Or, the ratio may depend on the proliferation of the cells (proliferation of the cells ultimately affects the ratio). The spheroids/embryoid bodies may be pre-fabricated or generated spontaneously in preparation. In some embodiments, the ratio of enhancement cells to seeded cells or ECM-generating cells is from 1:10 to 10:1. In some embodiments, the ratio of enhancement cells to seeded cells or ECM-generating cells is from 1:5 to 5:1. In some embodiments, the ratio of enhancement cells to seeded cells or ECM-generating cells is from 1:20 to 20:1. In some embodiments, the ratio of enhancement cells to seeded cells or ECM-generating cells is from 1:25 to 25:1. In some embodiments, the ratio of enhancement cells to seeded cells or ECM-generating cells is from 1:50 to 50:1. In some embodiments, the ratio of enhancement cells to seeded cells or ECM-generating cells is from 1:100 to 100:1. The present invention is not limited to the aforementioned ratios.

The tissue compositions of the present invention may further comprise proliferative cells different from the ECM-generating cells, e.g., adherent proliferative cells, e.g., mesenchymal stem cells, pre-vascular cells, endothelial cells, progenitor cells, etc. Note, there is a specific microenvironment for each type of cell, Thus, each cell type used would likely provide a specific niche to encourage elopement or integration of additional cell types.

As previously discussed, the seed cells may be human inducible pluripotent stem cell-derived cardiomyocytes or cardiomyocytes. In such a tissue composition, the cardiomyocytes can develop and spontaneously contract, e.g., in a synchronized manner. The cardiomyocytes allow for electrical signal propagation. In some embodiments, the scaffold directionalizes contractions. As previously discussed, in some embodiments, the fibers of the scaffold form grooves, and the grooves may help the scaffold directionalize cell seeding or contractions.

In the example with cardiomyocytes, in certain embodiments, the cardiomyocytes contract at a rate from 0 beats/min to 30 beats/min, 20 beats/min to 80 beats/min, 30 beats/min to 50 beats/min, 30 beats/min to 200 beats/min, 40 beats/min to 270 beats/min, 20 beats/min to 300 beats/min, 40 beats/min to 80 beats/min, etc. Note that factors such as but not limited to temperature, time post cryopreservation, lime post-seeding, etc. may influence beat rate. It is possible for the beat rate to be zero or to fluctuate during culture.

The seed cells may be present at a particular ratio with respect to the ECM-generating cells. The exact ratio may depend on the cell type of the seed cells. For example, in some embodiments, the ratio of seed cells to ECM-generating cells is from 1.10 to 10:1. In some embodiments, the ratio of seeded cells to ECM-generating cells is 1:1, 1:2, 1:3, 1:4, 1:5, 1:6, 1:7, 1:8, 1:9, 1:10, etc. In some embodiments, the ratio of seed cells to ECM-generating cells is from 1:5 to 5:1. In some embodiments, the ratio of seed cells to ECM-generating cells is from 1:20 to 20:1. In same embodiments, the ratio of seed cells to ECM-generating cells is from 1:25 to 25:1. In some embodiments, the ratio of seed cells to ECM-generating cells is from 1:50 to 50:1. In some embodiments, the ratio of seed cells to ECM-generating cells is from 1:100 to 100:1. In some embodiments, in a scaffold such as the hollow one of FIG. 3D, the ratio of seeded cells to ECM-generating cells is 1:1. Likewise, the amount of seed cells that are deposited on the tissue composition may depend on the cell type of the seed cell. In some embodiments, the seed cells are seeded to have a final density from 0.5×10⁶ cells/cm² to 5×10⁵ cells/cm². In some embodiments, the seed cells are seeded to have a final density from 1×10⁴ cells/cm² to 1×10⁷ cells/cm². In some embodiments, the seed cells are seeded to have a final density from 1×10² cells/cm² to 1×10⁷ cells/cm².

The arrangement of the seed cells may be a layer. Or, the arrangement of the seed cells on the scaffold may be in bundles, aggregates or groups of closely packed cells such as embryoid bodies, cardiospheres, etc. (or combinations of layers and bundles). The cell bundles may be of various sizes and may be mixed with single or layered cells. In certain embodiments, the tissue composition features from 3 to 500 cell layers.

In some embodiments, cell bundles (e.g., embryoid bodies) are seeded, e.g., cells are pre-clustered prior to seeding. In some embodiments, cells may form said bundles or aggregates after the seeding process. Note cells in such bundles (embryoid bodies) may exhibit their own microenvironment.

### Tissue Composition Features and Variations

In certain embodiments, the tissue composition comprises a biomaterial (e.g., a scaffold alone, a scaffold with seeded cells without ECM-generating cells, a scaffold with seeded cells and ECM-generating cells, a scaffold with ECM-generating cells, etc.) and a proliferative cell population. For example, the tissue composition may comprise a scaffold and ECM (with or without ECM-generating cells) and a proliferative cell population. In certain embodiments, the tissue composition comprises a biomaterial and a non-proliferative cell population. For example, the tissue composition may comprise a scaffold and ECM (with or without ECM-generating cells) and a non-proliferative cell population. The tissue compositions may have layers of cells from 3-500 cell layers thick, however the present invention is not limited to this configuration or range of cell layers. Tissue compositions herein may be cultured with factors (e.g., FGF), other proliferative cytokines, growth factors, or a combination thereof to achieve a desired thickness.

The biomaterial may be absorbable, non-absorbable, or a combination thereof. The biomaterial may feature synthetically-derived material, biologically-derived material, or a combination thereof. The biomaterial may comprise ECM-generating cells (e.g., fibroblasts) pre-seeded. In certain embodiments, the biomaterial does not comprise ECM-generating cells (e.g., fibroblasts), In certain embodiments, the biomaterial comprises pores. In certain embodiments, the biomaterial does not comprise pores. In certain embodiments, the biomaterial may feature ligands, antibodies, magnetic based particles, the like, or a combination thereof for attracting cells to the biomaterial. In certain embodiments, the culture plate, bioreactor, or other material used for producing the tissue composition may feature an anti-adhesion material on at least a portion of its surface for deterring cells from adhering to the surface and instead attaching to the biomaterial.

Non-limiting examples of proliferative cells include cardiac progenitors, fibroblasts, mesenchymal stem cells (MSCs), other progenitor cells (e.g., skeletal muscle progenitor cells, smooth muscle progenitor cells, neural progenitor cells, liver progenitor cells, etc.), and the like. Non-limiting examples of non-proliferative cells include cardiomyocytes, neural cells, pancreas cells, and the like.

Tissue compositions of the present invention, such as those made with proliferative cells, may be induced to continue to proliferate once they are seeded on the scaffold. Specific compositions (e.g., growth factors, peptides, etc.) may be used for this process. Tissue compositions of the present invention, such as those made with proliferative cells, may be allowed to proliferate on the construct and at some specified time be induced to differentiate into a specific cell type. For example, in a tissue composition comprising cardiac progenitors, the cardiac progenitors may be promoted to differentiate once the appropriate factor is introduced.

In certain embodiments, the tissue compositions (e.g., those made with proliferative cells described above) may be seeded with another cell population, e.g., endothelial cell population, cardiomyocyte population, mesenchymal stem cell (MSC) population, etc.

The tissue compositions herein may be constructed in various ways. For example, in certain embodiments, cell sheets or spheroids are generated and then subsequently transferred to the biomaterial. Cell sheets may be produced in a variety of ways. For example, cell sheets may be produced by seeding cells on a temperature sensitive plate, a low adhesion plate, or a plate with a composition (e.g., ligand) that allows for detachment of cells or tissue at a select time. Temperature-sensitive plates are designed to release adherent cells when placed at a particular temperature (e.g., between 20-25° C.). When the seeded cells have reached their appropriate confluency and/or morphology, the cells can be disassociated from the plate (e.g., temperature-sensitive plate, low adhesion plate, plate with ligand, etc.) as a sheet of cells and subsequently transferred to the biomaterial. Spheroids may be produced, for example, using centrifugation techniques, low adhesion plates, orbital shaking, etc. The spheroids can be pelleted and then seeded on to the biomaterial.

Without wishing to limit the present invention to any theory or mechanism, it is believed that the use of cell sheets or spheroids can increase the seeding efficiency of the tissue composition. This may be advantageous when working with an expensive and/or non-proliferative cell type.

The present invention also features tissue compositions constructed by seeding cells on a particular surface and subsequently adhering a biomaterial (e.g., a scaffold alone, a scaffold with seeded cells without ECM-generating cells, a scaffold with seeded cells and ECM-generating cells, a scaffold with ECM-generating cells, etc.) to the cells. Similar tissue compositions may be constructed by first adhering the biomaterial to the plate and then seeding cells. In certain embodiments, the tissue composition features layers of biomaterial and cells. Methods for adhering the biomaterial to the seeded cells may include but are not limited to centrifugation. In certain embodiments, the biomaterial comprises one or more components (e.g., ligands, etc.) for attracting the seeded cells. In certain embodiments, the seeded cells comprise one or more components (e.g., ligands) for attracting the biomaterial.

The surface may be a culture dish or culture surface with features that allow for removal of the tissue composition. For example, in some embodiments, the surface is a temperature-sensitive culture plate. The temperature-sensitive plate is designed to release adherent cells (e.g., the seeded cells of the tissue composition) when it is placed at a particular temperature (e.g., between 20-25° C.).

In certain embodiments, the plate comprises an attachment component for temporarily attaching the cells and/or biomaterial to the plate. For example, in certain embodiments the attachment component is a tuned liposome (e.g., gold-coated liposomes) with a ligand such as RGD. The ligand attaches the gold liposomes to the plate, and the cells and/or biomaterial attaches to the liposomes. When the tissue composition is ready for harvesting, the gold-coated liposomes can be activated with resonant light to open and thereby detach the tissue composition from the plate. The present invention is not limited to gold-coated liposomes. Without wishing to limit the present invention to any theory or mechanism, it is believed that one advantage to using an attachment component (such as the gold-coated liposomes) is it allows for constructing a tissue composition in a particular shape by patterning the attachment component (e.g., gold-coated liposomes) on the plate in that shape. The attachment components may also allow for stability and controlled release of cells and certain material that may be contained within the liposomes, e.g., factors required for growth and/or differentiation.

The present invention also features methods for reducing metabolic rate of tissue compositions. For example, the present invention features methods for reducing beat rates of tissue compositions, e.g., tissue compositions featuring cardiomyocytes. The methods may feature introducing a drug to the tissue composition that reduces the beat rate to a particular desired beat rate. In certain embodiments, the methods feature temperature control. In certain embodiments, the beat rate is reduced for the purpose of storage and/or stability, e.g., stability during transport. The methods for reducing the beat rate of a tissue composition may be applied to any appropriate tissue composition herein, e.g., tissue compositions featuring a scaffold, ECM, ECM-generating cells, and cardiomyocytes; tissue compositions featuring cells sheets with or without a scaffold; etc.

Furthermore, the present invention features tissue compositions (e.g., any appropriate tissue composition herein, other contractile grafts, etc.) with a beat rate from 10-20 bpm. The present invention also features tissue compositions (e.g., any appropriate tissue composition herein, other contractile grafts, etc.) with a beat rate from 20-30 bpm. The present invention also features tissue compositions (e.g., any appropriate tissue composition herein, other contractile grafts, etc.) with a beat rate from 10-30 bpm. The present invention also features tissue compositions (e.g., any appropriate tissue composition herein, other contractile grafts, etc.) with a beat rate from 30-40 bpm. The present invention also features tissue compositions (e.g., any appropriate tissue composition herein, other contractile grafts, etc.) with a beat rate from 20-40 bpm. The present invention also features tissue compositions (e.g., any appropriate tissue composition herein, other contractile grafts, etc.) with a beat rate from 40-50 bpm. The present invention also features tissue compositions (e.g., any appropriate tissue composition herein, other contractile grafts, etc.) with a beat rate from 30-50 bpm. The present invention also features tissue compositions (e.g., any appropriate tissue composition herein, other contractile grafts, etc.) with a beat rate from 0-50 bpm. The present invention also features tissue compositions (e.g., any appropriate tissue composition herein, other contractile grafts, etc.) with a beat rate from 0-100 bpm. Without wishing to limit the present invention to any theory or mechanism, it is believed that a low beat rate (e.g., a beat rate from 10-50 bpm) may be advantageous because tissue compositions with a low beat rate would have a lower metabolic burden as compared to those with a high beat rate, and the lower metabolic burden may help extend the shelf life of the tissue composition (e.g., the amount of time that the graft could be set at room temperature before being implanted). The lower metabolic burden may also be beneficial in an ischemic environment because the tissue compositions would require fewer nutrients to be healthy and functional.

Methods for modulating the beat rate of a tissue composition include but are not limited to the use of beta blockers or other exogenous factors.

The tissue compositions of the present invention are constructed to withstand short-term and/or long-term storage, e.g., cryopreservation. Cryopreservation may refer to a temperature of −80° C. to −196° C. or −90° C. to 196° C. The ability to cryopreserve the tissue composition helps allow for stocking tissues until use on demand as well as for transporting the tissue compositions from one location to another.

The tissue compositions may also be constructed to withstand certain lengths of time at room temperature (or a temperature below 37° C.). The ability to remain viable at a temperature below 37° C. for certain lengths of time may be beneficial for instances when the tissue composition is out of the incubator prior to use. As an example, a tissue composition may be exposed to room temperature for a lengthy period of time when it is removed from the incubator, brought to an operating room for use in an implantation process, but is not implanted immediately.

### Properties of the Tissue Composition

The engineered tissue compositions of the present invention (e.g., featuring cardiomyocytes) can be evaluated fore one or more mechanical parameters, electrophysiological parameters, chemical parameters, biochemical parameters (growth factors, metabolites, ion channels, etc.), or a combination thereof. Mechanical parameters or electrophysiological parameters may include but are not limited to contraction rate, contraction/relaxation velocity, force of contraction-paced, force of contraction—not paced, displacement velocity, displacement force, directionality of impulse, velocity of impulse, field potential, amplitude, capture threshold, chronotropic response, activation sequence after stimulation, functional gap junction formation, response to electrical pacing, field potential amplitude, conduction velocity, propagation patterns, gap junction analysis, or a combination thereof.

Likewise, the engineered tissue composition (e.g., featuring cardiomyocytes) can be subjected to multi-electrode array mapping for real-time electrophysiology measurements. Contraction rate, systolic/diastolic displacement, systolic contraction velocity, and/or diastolic relaxation velocity may be detected with a microscope. As previously discussed, the cardiomyocytes can be paced. Pacing may be achieved by external field stimulation applied.

The tissue composition, for example a tissue composition comprising cardiomyocytes, may be constructed to have a particular beat rate. In certain embodiments, the beat rate is from 0-100 beats per minute (bpm). In certain embodiments, the beat rate is from 10 to 30 bpm. In certain embodiments, the beat rate is from 20 to 40 bpm. In certain embodiments, the beat rate is from 30 to 60 bpm. In certain embodiments, the beat rate is from 40 to 70 bpm. In certain embodiments, the beat rate is from 50 to 80 bpm. In certain embodiments, the beat rate is from 60 to 90 bpm. In certain embodiments, the beat rate is from 70 to 100 bpm. The present invention is not limited to the aforementioned examples of beat rates.

The mechanical properties of the engineered tissue composition (e.g., featuring cardiomyocytes) may depend on the scaffold material used. For example, displacement, strain percentage, displacement velocity, etc., may all depend on the material of the scaffold. In some embodiments, the engineered tissue composition (e.g., featuring cardiomyocytes) has a voltage amplitude across the engineered tissue composition from 0.1 mV to 1 mV with inter-electrode spacing of 1 mm-1.5 cm.

The tensile strength of the tissue composition can be determined by its composition, e.g., the percentage of scaffold, ECM, cells, etc.

In certain embodiments, the relative expression of a marker can be evaluated to determine the amount of a particular cell type of interest (e.g., cardiomyocyte, skeletal muscle cell, smooth muscle cell, etc.) relative to the ECM-generating cells (e.g., fibroblast), The ratio of the cell type of interest to the ECM-generating cells will change over time based on the changes of the tissue composition, e.g., if the cells of interest proliferate, if certain cell populations die, if cells differentiate over time, etc. Non-limiting examples of markers that may be evaluated include CD90, vimentin, FSP-1, collagen I, alpha-SMA, HSP47, etc.

### Platforms

The present invention also features platforms with the tissue compositions of the present invention. For example, the present invention features a single-well plate (with a single well), wherein an engineered tissue composition of the present invention is deposited in the well therein. The present invention also features multi-well plate with two or more wells, wherein an engineered tissue composition of the present invention is deposited in at least one well therein. The multi-well plate may comprise two wells, four wells, six wells, eight wells, 12 wells, 24 wells, 48 wells, 96 wells, more than 96 wells, from 2 to 12 wells, from 12 to 24 well, from 24 to 48 wells, from 48 to 96 wells, etc. The present invention also features platforms for the engineered tissue composition comprising wells within wells, troughs, or any other appropriate culture apparatus such as a tube, tray, etc. The present invention is not limited to a culture dish as a platform for culturing, maintaining, and/or storing the tissue compositions.

The present invention also features closed system platforms for producing, maintaining, and/or storing the tissue compositions herein. For example, the closed system may feature an encapsulation with the tissue composition, e.g., the scaffold and ECM and optionally other components as discussed herein, housed therein. Media can be exchanged in the closed system in a sterile manner. The tissue compositions can also be frozen in the encapsulation and thawed when ready. In certain embodiments, more than one tissue composition can be housed in the encapsulation or multiple encapsulations can be connected together to create a new encapsulation. For example, in some embodiments, up to 6 tissue compositions are housed in the encapsulation. In some embodiments, up to 10 tissue compositions are housed in the encapsulation. In some embodiments, up to 20 tissue compositions are housed in the encapsulation. In some embodiments, more than 20 compositions are housed in the encapsulation.

### Cryopreservation and Reconstitution

The present invention features systems, methods, and compositions for cryopreservation and reconstitution of engineered tissue compositions.

The composition of the tissue is important to enable cryopreservation and reconstitution without damage of the tissue. The risks of damage from freezing and thawing are generally related to the expansion and contraction of the material as the temperature changes. Cells and tissues have high water content. As cells expand and contract the cells may be damaged from ice crystal formation or rupture. The use of cryopreservation solutions can help prevent ice crystal formation and damage. Also, the cellular material in the tissue may be protected by using materials that can support the cell so that it resists damage during the expansion and contraction due to temperature changes. The material acts as a composite with the cells to create a robust tissue. The material supports the cell membrane to lessen the impact of the change in temperature and thus resist damage. Extracellular matrix and/or biomaterial can be used as a support for cryopreservation and reconstitution. The material that is used as a support must be able to be frozen without fracture or other damage. The material used as a support should be of appropriate stiffness/elasticity, strength and porosity as well as appropriate % volume of the tissue. Too stiff or too soft, the tissues will not be able to be cryopreserved.

Any of the engineered tissue compositions disclosed herein may be considered for cryopreservation and/or reconstitution. For example, in some embodiments, the scaffold is a knitted biomaterial. In some embodiments the scaffold is a woven, spun, electrospun, layered, laminated, or printed. In some embodiments, the scaffold or the engineered tissue composition has a maximum stiffness from 0.5 N/mm to 4 N/mm. In some embodiments the scaffold has a stiffness from 2 N/mm to 3.5 N/mm. In some embodiments, the scaffold or the engineered tissue composition has a tensile strength from 0.64-0.15 MPa (or 0.04 to 0.1 MPa), In some embodiments the scaffold or engineered tissue composition has a tensile strength from 0.05 to 0.1 MPa. As previously discussed, biomaterials used in the present invention may be bioabsorbable or non-absorbable (or a combination of bioabsorbable and non-absorbable materials may be used.

Reconstitution may be performed by rapid warming of the tissue and reintroducing the tissue to culture conditions for a period of time such as 4 hours, 8 hours, 24 hours or 48 hours. For example the tissue may be taken from −80 degrees C. to a 37 degree C. water bath, where it is held from 1-5 minutes, then transferred to a culture incubator at 37 degrees C. where they are cultured for approximately 24 to 48 hours. Additional reconstitution following thaw may not be required.

Where additional reconstitution following thaw is required, the reconstitution may be accelerated to a time taking less than 48 hours or less than 24 hours. Reconstitution may be accelerated by temperature changes or temperature cycling (e.g. cycling temperature between 35 and 39 degrees C. one time, two times, three times, etc.) Reconstitution may also be accelerated by light, ultrasonic, neural, electrical, electromagnetic, physical or chemical means. For example, an electrical stimulus may be applied to the tissue or the medium containing the tissue to activate the tissue. Alternatively a physical force such as stretch or compression could be applied or an anti-gravity environment could be applied. Alternatively, specific wavelengths of electromagnetic radiation may be used to accelerate the reconstitution by activation of biological processes. Also, through chemical means the tissue may be stimulated to speed reconstitution and functionality/potency such as through the use of nutrients or drugs activating the natural biological processes in the cell. For example, catecholamines or beta adrenergic agonist or other drugs working through alpha or beta adrenergic receptors may be used to activate cardiac cells.

An example of a cryopreservation method of the present invention may include one or a combination of the following features: a starting temperature of about 4° C., nucleation at a temperature from −20° C. to −40° C., a final temperature from −80° C. to −90° C., and storage from −80° C. or −196° C.

As previously discussed, in some embodiments, the cryopreservation process is performed in a closed system. In some embodiments, the cryopreservation process is performed in an open system. In some embodiments, the thawing/reconstitution process is performed in a closed system. In some embodiments, the thawing/reconstitution process is performed in an open system.

The tissue compositions herein may be defined as comprising (i) a scaffold and seeded cells on or within the scaffold; (ii) comprising a scaffold and ECM-generating cells disposed on or within the scaffold (or both on and within the scaffold); (iii) a scaffold, ECM material disposed on the scaffold or within the scaffold (or both on and within the scaffold), and ECM-generating cells disposed on or within the scaffold (or both on and within the scaffold); (iv) a scaffold, ECM material disposed on the scaffold or within the scaffold (or both on and within the scaffold), and seeded cells in or on the scaffold with the ECM (or both in and on the scaffold with the ECM); or (v) a scaffold, ECM material disposed on the scaffold or within the scaffold (or both on and within the scaffold); ECM-generating cells disposed on or within the scaffold (or both on and within the scaffold), and seeded cells in and/or on the scaffold along with the ECM-generating cells and ECM.

For each of the aforementioned embodiments of tissue compositions, the tissue compositions may be functional after a cryopreservation-thaw cycle. For certain embodiments, the term ‘functional after a cryopreservation-thaw cycle’ may refer to one or a combination of: the tissue composition maintaining its physical integrity after the cryopreservation-thaw cycle: the tissue composition exhibiting cell-cell communication after the cryopreservation-thaw cycle; the tissue composition exhibiting synchronous contractions after the cryopreservation-thaw cycle; the tissue composition (e.g., cells of the tissue composition) secreting a neurotransmitter after the cryopreservation-thaw cycle; the tissue composition (e.g., cells of the tissue composition) secreting a hormone after the cryopreservation-thaw cycle; the tissue composition (e.g., cells of the tissue composition) secreting an enzyme after the cryopreservation-thaw cycle; the tissue composition (e.g., cells of the tissue composition) secreting a cytokine after the cryopreservation-thaw cycle; the tissue composition (e.g., cells of the tissue composition) secreting a growth factor after the cryopreservation-thaw cycle; the tissue composition (e.g., cells of the tissue composition) secreting RNA alter the cryopreservation-thaw cycle; etc.

Table 1 below lists examples of markers that may be used to determine if the tissue composition is functional after the cryopreservation-thaw cycle. For example, a tissue composition designed for heart tissue may be evaluated to ensure the tissue composition has appropriate synchronous contractions following the cryopreservation-thaw cycle. Or, that tissue composition may be tested to determine if it is secreting an appropriate amount of IGF-1 In some embodiments, the tissues compositions are tested for more than one marker. The present invention is not limited to the markers for tissue functionality listed below or described herein.

TABLE 1

| Tissue | |
|---|---|
| Composition | Markers for Determining Tissue Functionality Post-Thaw |
| Heart | Synchronous contraction, Connexin-43, Natriuretic peptides |
| | (ANP, BNP), Alpha- or beta- myosin heavy chain, Alpha |
| | actinin, Fibroblast growth factor, IGF-1, Angiopoietin |
| Liver | Prostanoids, Nitric oxide, Endothelin, TNF-alpha, |
| | Interleukins, TGF-beta, PDGF, HGF, EGF, Albumin |
| | secretion, Bile secretion, Expression of hepatocyte |
| | growth factor, Fibroblast growth factor, IGF-1, |
| | Angiopoietin, IL-6 |
| Pancreas | Insulin secretion, Fibroblast growth factor, IGF-1, |
| | Angiopoietin, Connexions, Cadherins, Eph Receptors, |
| | Ephrine ligands |
| CNS | Production of electrical currents from neurons, |
| tissue | Fibroblast growth factor, IGF-1, Angiopoietin, Calcium, |
| | Sonic hedgehog protein, Cadherins, Synapses |
| Intestinal | Formation of tight junctions joining the cells of the |
| epithelium/ | epithelial layer, Fibroblast growth factor, IGF-1, |
| mucosa | Angiopoietin, Cytokines, TGF-beta, IL-10, Secretion of |
| | mucous, Antimicrobial peptides, Gastric acids (CCK, |
| | gastrin, somatostatin, etc.) |
| Skeletal | Connexins, Stimulated contraction, IGF-1, TGF-beta, |
| Muscle | Myostatin, Myokines, Musculin, Calcium |
| Vascular | VEGF, Platelet derived growth factor, Ang-1, Nitric |
| | oxide, eNOS expression, endothelin, Kinases, Cadherins |

For each of the embodiments of tissue compositions described herein, following the cryopreservation-thaw cycle, the tissue compositions may be tested to determine whether the amount of hormone, growth factor, cytokine, enzyme, RNA, etc. being secreted is adequate.

In certain embodiments, the amount of hormone secreted is not less than 10% of the amount of hormone secreted by cells of the tissue composition prior to the cryopreservation-thaw cycle, e.g., is not less than 9%, 8%, 7%, 6%, 5%, 4%, 3%, 2%, 1%. In certain embodiments, the amount of hormone secreted is not less than 20% of the amount of hormone secreted by cells of the tissue composition prior to the cryopreservation-thaw cycle, e.g., not less than 19%, 18%, 17%, 16%, 15%, 14%, 13%, 12%, 11%, etc. In certain embodiments, the amount of hormone secreted is not less than 30% of the amount of hormone secreted by cells of the tissue composition prior to the cryopreservation-thaw cycle, e.g., not less than 29%, 28%, 27%, 26%, 25%, 24%, 23%, 22%, 21%, etc. In certain embodiments, the amount of hormone secreted is greater than the amount of hormone secreted by cells of the tissue composition prior to the cryopreservation-thaw cycle.

In certain embodiments, the amount of neurotransmitter secreted is not less than 10% of the amount of neurotransmitter secreted by cells of the tissue composition prior to the cryopreservation-thaw cycle, e.g., is not less than 9%, 8%, 7%, 6%, 5%, 4%, 3%, 2%, 1%. In certain embodiments, the amount of neurotransmitter secreted is not less than 20% of the amount of neurotransmitter secreted by cells of the tissue composition prior to the cryopreservation-thaw cycle, e.g., not less than 19%, 18%, 17%, 16%, 15%, 14%, 13%, 12%, 11%, etc. in certain embodiments, the amount of neurotransmitter secreted is not less than 30% of the amount of neurotransmitter secreted by cells of the tissue composition prior to the cryopreservation-thaw cycle, e.g., not less than 29%, 28%, 27%, 26%, 25%, 24%, 23%, 22%, 21%, etc. In certain embodiments, the amount of neurotransmitter secreted is greater than the amount of neurotransmitter secreted by cells of the tissue composition prior to the cryopreservation-thaw cycle.

In certain embodiments, the amount of enzyme secreted is not less than 10% of the amount of enzyme secreted by cells of the tissue composition prior to the cryopreservation-thaw cycle. e.g., is not less than 9%, 8%, 7%, 6%, 5%, 4%, 3%, 2%, 1%. In certain embodiments, the amount of enzyme secreted is not less than 20% of the amount of enzyme secreted by cells of the tissue composition prior to the cryopreservation-thaw cycle, e.g., not less than 19%, 18%, 17%, 16%, 15%, 14%, 13%, 12%, 11%, etc. In certain embodiments, the amount of enzyme secreted is not less than 30% of the amount of enzyme secreted by cells of the tissue composition prior to the cryopreservation-thaw cycle, e.g., not less than 29%, 28%, 27%, 26%, 25%, 24%, 23%, 22%, 21%, etc. In certain embodiments, the amount of enzyme secreted is greater than the amount of enzyme secreted by cells of the tissue composition prior to the cryopreservation-thaw cycle.

In certain embodiments, the amount of growth factor secreted is not less than 10% of the amount of growth factor secreted by cells of the tissue composition prior to the cryopreservation-thaw cycle, e.g., is not less than 9%, 8%, 7%, 6%, 5%, 4%, 3%, 2%, 1%. In certain embodiments, the amount of growth factor secreted IS not less than 20% of the amount of growth factor secreted by cells of the tissue composition prior to the cryopreservation-thaw cycle, e.g., not less than 19%, 18%, 17%, 16%, 15%, 14%, 13%, 12%, 11%, etc. In certain embodiments, the amount of growth factor secreted is not less than 30% of the amount of growth factor secreted by cells of the tissue composition prior to the cryopreservation-thaw cycle, e.g., not less than 29%, 28%, 27%, 26%, 25%, 24%, 23%, 22%, 21%, etc. In certain embodiments, the amount of growth factor secreted is greater than the amount of growth factor secreted by cells of the tissue composition prior to the cryopreservation-thaw cycle.

In certain embodiments, the amount of cytokine secreted is not less than 10% of the amount of growth factor secreted by cells of the tissue composition prior to the cryopreservation-thaw cycle. e.g., is not less than 9%, 8%, 7%, 6%, 5%, 4%, 3%, 2%, 1%. In certain embodiments, the amount of cytokine secreted is not less than 20% of the amount of growth factor secreted by cells of the tissue composition prior to the cryopreservation-thaw cycle, e.g., not less than 19%, 18%, 17%, 16%, 15%, 14%, 13%, 12%, 11%, etc. In certain embodiments, the amount of cytokine secreted is not less than 30% of the amount of growth factor secreted by cells of the tissue composition prior to the cryopreservation-thaw cycle, e.g., not less than 29%, 28%, 27%, 26%, 25%, 24%, 23%, 22%, 21%, etc. In certain embodiments, the amount of cytokine secreted is greater than the amount of cytokine secreted by cells of the tissue composition prior to the cryopreservation-thaw cycle.

In certain embodiments, the amount of RNA secreted is not less than 10% of an amount of growth factor secreted by cells of the tissue composition prior to the cryopreservation-thaw cycle, e.g., is not less than 9%, 8%, 7%, 6%, 5%, 4%, 3%, 2%, 1%. In certain embodiments, the amount of RNA secreted is not less than 20% of an amount of growth factor secreted by cells of the tissue composition prior to the cryopreservation-thaw cycle; e.g., not less than 19%, 18%, 17%, 16%, 15%, 14%, 13%, 12%, 11%, etc. In certain embodiments, the amount of RNA secreted is not less than 30% of an amount of growth factor secreted by cells of the tissue composition prior to the cryopreservation-thaw cycle, e.g., not less than 29%, 28%, 27%, 26%, 25%, 24%, 23%, 22%, 21%, etc. In certain embodiments, the amount of RNA secreted is greater than the amount of RNA secreted by cells of the tissue composition prior to the cryopreservation-thaw cycle.

In certain embodiments, the ECM-generating cells are live, dead, or a portion of the ECM-generating cells are dead. In certain embodiments, the ECM-generating cells are seeded on the scaffold first and subsequently produce the ECM. In certain embodiments, the ECM is deposited on the scaffold prior to seeding of the ECM-generating cells. In certain embodiments, the seeded cells are stem cells, embryonic stem cells, embryonic stem cell-derived cells, induced pluripotent stem cell-derived cells, adult stem cells, progenitor cells, cardiac cells, a skeletal muscle cells, smooth muscle cells, liver cells, pancreatic cells, lung cells, bone cells, umbilical cord cells, endothelial cells, central nervous system cells, gastrointestinal cells, endocrine cells, salivary cells, mesenchymal stem cells, fibroblast cells, endothelial cells, epithelial cells, or paracrine cells. In certain embodiments, the seeded cells are seeded as spheroids or in the form of a cell sheet, in the form of a gel, or in the form of a foam.

The present invention also features a workflow system for an engineered tissue composition. In certain embodiments, the workflow system comprises culturing the engineered tissue composition (any tissue composition according to the present invention); cryopreserving the engineered tissue composition; and reconstituting the engineered tissue composition, wherein the engineered tissue composition is functional after a cryopreservation-thaw cycle (as described herein); wherein the system is a closed system. In some embodiments, the system provides a sterile environment for culturing, cryopreserving, and reconstituting the engineered tissue composition.

The present invention also features a method of enhancing a function of an engineered tissue composition, the function may refer to any of the aforementioned functions (e.g., secretion of an enzyme, hormone, cytokine, RNA, growth factor; exhibiting cell-cell communication; etc.). In certain embodiments, the method comprises cryopreserving the engineered tissue composition and reconstituting the engineered tissue composition, wherein the engineered tissue composition has enhanced function after the cryopreservation-thaw cycle, e.g., enhanced secretion of an enzyme, hormone, cytokine, RNA, growth factor, etc.

The present invention also features a method of enhancing a function of an engineered tissue composition, the function may refer to any of the aforementioned functions (e.g., secretion of an enzyme, hormone, cytokine, RNA, growth factor; exhibiting cell-cell communication; etc.). In certain embodiments, the method comprises chilling the engineered tissue composition to a temperature below 37° C. (e.g., a temperature from −196° C. to less than 37° C., a temperature from 30 to 36° C., from 25 to 30° C., from 25 to 35° C., from 20 to 30° C., from 15 to 30° C., from 15 to 25° C., from 15 to 20° C., from 10 to 25° C., from 10 to 20° C., from 10 to 15° C., from 5 to 10° C., from 5 to 15° C., from 0 to 15° C., from 0 to 5° C., from 1 to 4° C., from −10 to 0° C., from 190 to 0° C., from 0 to 36° C., from 0 to 25° C., from 0 to 10° C., from −20 to −10° C., from −20 to 0° C. from −30 to 0° C., from −40 to −20° C., from −80 to 0° C., from −190 to −80° C. etc.) and reconstituting the engineered tissue composition, wherein the engineered tissue composition has enhanced function after being chilled, e.g., enhanced secretion of an enzyme, hormone, cytokine, RNA, growth factor, etc. The tissue composition may be held at the chilled temperature or a combination of chilled temperatures for a period of time, e.g., for minutes or hours, e.g., for a time period from 10 minutes to 72 hours, 1-72 hours, 2 to 24 hours, 4 to 12 hours, 4 to 8 hours, 6 to 12 hours 8 to 20 hours, 10 to 24 hours, 24 to 48 hours, 48 to 72 hours, etc.

The present invention also features a method of enhancing a function of an engineered tissue composition, the function may refer to any of the aforementioned functions (e.g., secretion of an enzyme, hormone, cytokine, RNA, growth factor; exhibiting cell-cell communication; etc.), In certain embodiments, the method comprises incubating the engineered tissue composition at a temperature above 37° C. (e.g., 38° C., 39° C., 40° C., 41° C., 42° C., 43° C., 44° C., 45° C., 46° C., 47° C., 48° C., 49° C., 50° C., a temperature from 38° C. to 50° C., from 38° C. to 42° C., from 39° C. to 42° C., etc.) and reconstituting the engineered tissue composition, wherein the engineered tissue composition has enhanced function after being incubated, e.g., enhanced secretion of an enzyme, hormone, cytokine, RNA, growth factor, etc. The hyperthermia may be used prior to cryopreservation, following cryopreservation, or independent of cryopreservation. The duration of the hyperthermia may be seconds to hours, e.g. 10 seconds to 4 hours, or 30 seconds to 30 minutes, etc.

### Example 1

Example 1 discloses a method of cryopreserving an engineered tissue composition in an open system, as well as method of thawing and reconstituting the engineered tissue composition.

Cryopreservation: Tissues (e.g., 1.6 cm diameter) are removed from their culture at 37° C. and transferred to 1.5 ml vials containing 1 ml of chilled (4° C.) cryopreservation solution. Tissues are placed into the vials and completely submerged using forceps. The vials are then placed into a pre-chilled 2-8° C. ethanol jacketed or foam insulated container and placed in a −80° C. freezer. After 5 hours the vials can be stored at −80° C. or transferred to −196° C. until needed. In an alternative method, the vials are then placed into a pre chilled 2-8° C. ethanol jacketed or foam insulated container and placed in 2-8° C. for 20 min. The container containing the vials is then transferred to −80° C. After ˜20 minutes, the container is tapped lightly on the sides to support ice nucleation. Without wishing to limit the present invention to any theory or mechanism, it is believed that nucleation can be initiated by force, seeding, chemical, electrofreezing, mechanical, shock cooling, pressure, etc. The container is then placed back in the −80° C. for up to 5 hours and either stored at −80° C. or transferred to −196° C.

Thaw and Reconstitution: The vials are transferred to a 37° C. water bath and submerged, leaving the caps above the water. They are warmed for 1-5 min (e.g., 4 min) and then transferred to the biosafety hood and removed under sterile conditions using forceps. The tissues are then transferred to a 24 well plate containing 2 ml of warmed culture medium (DMEM, RPMI or similar) and may or may not be supplemented with B27, FBS, albumin or similar. The cultures may also be supplemented with 10 μM ROCK inhibitor Y-27632 to limit apoptosis. The tissues are cultured for 24 hrs and the medium changed. The medium is then changed every 24 hours until the tissues are used, e.g., 48 hours. In certain embodiments, the reconstitution is achieved within 20 hours, IN some embodiments, reconstitution is achieved within 24-30 hours. In some embodiments, reconstitution is achieved within 48 hours.

### Example 2

Example 2 discloses a method of cryopreserving an engineered tissue composition in a closed system, as well as method of thawing and reconstituting the engineered tissue composition.

Cryopreservation: Tissues (e.g., 5.2 cm diameter) complete culture at 37° C. and are transferred to a closed environmental system (e.g., biobag, Origen CS50 cryobag. Charter medical FPFlex50B, etc.). In some embodiments, the bags are modified. For example, in some embodiments, the bags are cut along the base and sometimes a side, the grafts are then placed into the modified bag, which is then heat sealed (or similar as needed for the biobag material being used) to the environment to start the process of cryopreservation. The cryobag contains at least one and sometimes multiple luer ports in addition to spike ports. Using sterile technique, the luer is used to. Introduce 10-30 ml of chilled (4° C.) Biolife solutions CS10 (or similar) via a syringe. The cryobag is then placed in a chilled (4° C.) cooler until all tissues placed intro cryobags and prepared for freezing. Exposed luer lines are then all sealed with a tube welder and all cryobags transferred to a controlled rate freezer. The controlled rate freezer will then progress through a program to reduce the temperature to −80° C.

Thaw and Reconstitution: Tissues frozen at −196° C. are transferred to −80° C. for a minimum of 24 hrs or as long at 7 days prior to thaw. Without wishing to limit the present invention to any theory or mechanism, while the example methods herein do not feature transferring an engineered tissue composition from −196° C. directly to a 37° C. water bath, it may be possible to do so depending on the properties of the engineered tissue composition.

The cryobags are transferred to a 37° C. water bath, submerged, and warmed for 1-5 min. In some embodiments, the cryobags are flipped at one or more times during the thaw. The cryobags are then visually inspected until all ice is thawed and then transferred to the biosafety hood. One of the spike ports is accessed using sterile technique and the cryomedium is removed using a syringe. Using sterile technique, the tissue is washed and warmed medium is introduced, e.g., 5 ml and sometimes up to 10 ml (but no less than 3 ml) of warmed culture medium (DMEM, RPM or similar). The medium may or may not be supplemented with B27, FBS, albumin or similar. The cultures may also be supplemented with 10 μM ROCK inhibitor Y-27632. The tissues are then cultured in the cryobag for 24 hrs. The medium is then changed using sterile technique every 24 hrs until the tissues are used.

As previously discussed, in some embodiments, the cryopreservation process and/or the thaw/reconstitution process may be performed in a closed system. For example, in some embodiments, once the cryobag is thawed, a peristaltic pump may be used to remove all cryomedium. A second bag may be attached to the cryobag, e.g., the empty bag may be spiked into the cryobag, tube welded in place, connected using an aseptic connector (e.g., ReadyMate, (GE)), etc. The second bag may contain the desired culture medium (e.g., as described above) to reconstitute the tissue. The bag may then proceed down one of two paths for reconstitution.

Path 1: The cryobag containing the product and fresh culture medium may then be placed in a standard cell culture incubator where it could be maintained. Tissues may be cultured for less than 24 hours. Tissues may be cultured for 24 hours and the medium changed. The medium may then be changed every 24 hours until the tissues are used.

Path 2: The cryobags may be attached to a bioreactor (such as Xuri) where they are cultured until needed. The cultures may contain rocking action (2-8 rpm) with less rocking initially and more rocking later on. In some embodiments, the angle or rock is from 2-8 degrees. In some embodiments, perfusion or culture medium is added. Perfusion may be done continuously at 2-20 rpms or for periods of time. Once the reconstitution time has been completed the tissues are ready to use.

Without wishing to limit the present invention to any theory or mechanism, it is believed that the composition and the vessels are important for enabling reconstitution in a closed system.

Shipping: In some embodiments, the tissue may be shipped once they have been fully reconstituted. In some embodiments, the tissues may be reconstituted on site. Shipping may feature sealing shut the cryobag tubing and then shipping the cryobag to the surgical site. The surgical team may maintain the tissues at 37° C. flushing the cryobag with saline (e.g., 2×) before maintaining it at 37° C. for up to 2 hours. At the time of use, the saline may be removed from the bag using a syringe. The bag may then be cut open to expose the tissue, which is then removed and implanted as needed.

Alternative approaches such as HypoThermosol® can also be considered. HypoThermosol® is used to maintain products at 2-8° C. during shipping to enhance stability. This proprietary, optimized formulation mitigates temperature-induced molecular cell stress responses that occur during chilling and re-warning of biologics, intermediate products, and final cell products intended for research and clinical applications. HypoThermosol® FRS includes components that scavenge free radicals, provide pH buffering, oncotic/osmotic support, energy substrates, and ionic concentrations that balance the intracellular state at low temperatures. Across a broad spectrum of cell and tissue types, HypoThermosol® has proven much more effective in reducing post-preservation necrosis and apoptosis as compared to commercial and home-brew isotonic and extracellular formulations. This results in greatly extended shelf life and improved post-preservation viability.

### Example 3

Example 3 shows growth factor and cytokine expression levels of fresh vs thawed (previously cryopreserved) cardiac grafts (terminally differentiated cardiomyocytes co-cultured with fibroblasts) as assessed through ELISA assay (see Table 2 below). Data are mean+SE. * denotes statistical difference (p<0.05). N=3 for Fresh, n=3 for Thawed.

TABLE 2

| Agent | Fresh (pg/ml) | Thawed (pg/ml) |
|---|---|---|
| IGF-1 | 0.16 | 0.17 |
| VEGF | 1300 | 800* |
| FGF | 30 | 70 |
| HGF | 650 | 1200* |
| G-CSF | 1200 | 1600 |
| TGF-b | 750 | 800 |

The disclosures of the following U.S. patents are incorporated in their entirety by reference herein: U.S. Pat. App. No. 2018/0361025; U.S. Pat. No. 4,963,489; U.S. Pat. App. No. US2009/0269316; WO2013151755; WO2011102991; U.S. Pat. App. No. 2014/0178450; U.S. Pat. No. 8,802,144; WO2009102967; U.S. Pat. No. 9,119,831; WO2010042856; U.S. Pat. No. 2008/0075750, U.S. Pat. No. 9,587,222.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety. Although there has been shown and described the preferred embodiment of the present invention, it will be readily apparent to those skilled in the art that modifications may be made thereto which do not exceed the scope of the appended claims. Therefore, the scope of the invention is only to be limited by the following claims. Reference numbers recited in the claims are exemplary and for ease of review by the patent office only and are not limiting in any way. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting of”, and as such the written description requirement for claiming one or more embodiments of the present invention using the phrase “consisting of” is met.

What is claimed is:

1. A tissue composition comprising:
a. a scaffold; and
b. seeded cells on or within the scaffold;
wherein after a cryopreservation-thaw cycle the tissue composition exhibits cell-cell communication.

2. A tissue composition comprising:
a. a scaffold; and
b. seeded cells on or within the scaffold;
wherein after a cryopreservation-thaw cycle the tissue composition maintains its physical integrity.

3. A tissue composition comprising:
a. a scaffold; and
b. seeded cells on or within the scaffold;
wherein after a cryopreservation-thaw cycle, cells of the tissue composition secrete one or a combination of a hormone, an enzyme, a growth factor, a cytokine, or an RNA.

4. A tissue composition comprising:
a. a scaffold; and
b. seeded cells on or within the scaffold;
wherein after a cryopreservation-thaw cycle the tissue composition exhibits synchronous contractions.

5. The tissue composition of claim 3, wherein the amount of hormone secreted is not less than 10% of an amount of hormone secreted by cells of the tissue composition prior to the cryopreservation-thaw cycle.

6. The tissue composition of claim 3, wherein the amount of hormone secreted is not less than 20% of an amount of hormone secreted by cells of the tissue composition prior to the cryopreservation-thaw cycle.

7. The tissue composition of claim 3, wherein the amount of hormone secreted is greater than an amount of hormone secreted by cells of the tissue composition prior to the cryopreservation-thaw cycle.

8. The tissue composition of claim 3, wherein the amount of enzyme secreted is not less than 10% of an amount of enzyme secreted by cells of the tissue composition prior to the cryopreservation-thaw cycle.

9. The tissue composition of claim 3, wherein the amount of enzyme secreted is not less than 20% of an amount of enzyme secreted by cells of the tissue composition prior to the cryopreservation-thaw cycle.

10. The tissue composition of claim 3, wherein the amount of enzyme secreted is greater than an amount of enzyme secreted by cells of the tissue composition prior to the cryopreservation-thaw cycle.

11. The tissue composition of claim 3, wherein the amount of growth factor secreted is not less than 10% of an amount of growth factor secreted by cells of the tissue composition prior to the cryopreservation-thaw cycle.

12. The tissue composition of claim 3, wherein the amount of growth factor secreted is not less than 20% of an amount of growth factor secreted by cells of the tissue composition prior to the cryopreservation-thaw cycle.

13. The tissue composition of claim 3, wherein the amount of growth factor secreted is greater than an amount of growth factor secreted by cells of the tissue composition prior to the cryopreservation-thaw cycle.

14. The tissue composition of claim 3, wherein the amount of cytokine secreted is not less than 10% of an amount of growth factor secreted by cells of the tissue composition prior to the cryopreservation-thaw cycle.

15. The tissue composition of claim 3, wherein the amount of cytokine secreted is not less than 20% of an amount of growth factor secreted by cells of the tissue composition prior to the cryopreservation-thaw cycle.

16. The tissue composition of claim 3, wherein the amount of cytokine secreted is greater than an amount of growth factor secreted by cells of the tissue composition prior to the cryopreservation-thaw cycle.

17. The tissue composition of claim 3, wherein the amount of RNA secreted is not less than 10% of an amount of growth factor secreted by cells of the tissue composition prior to the cryopreservation-thaw cycle.

18. The tissue composition of claim 3, wherein the amount of RNA secreted is not less than 20% of an amount of growth factor secreted by cells of the tissue composition prior to the cryopreservation-thaw cycle.

19. The tissue composition of claim 3, wherein the amount of RNA secreted is greater than an amount of growth factor secreted by cells of the tissue composition prior to the cryopreservation-thaw cycle.

20. The tissue composition of any of claims 3 and 11-13, wherein the growth factor is one or a combination of IGF-1, VEGF, FGF, HGF, G-SCF, or TGF-b, angiopoietin-1, TBG-b3, PDGF-A, EPO, HBEGF, TGFa, Angiogenein, Angiopoientin-2, Endothelial Growth Factor, Leptin, PDGF-BB, SPARC, IL-6, IL-8, Interferon-gamma, IL-1a, IL-1b, IL-6, IL-8, Monocyte chemotactic protein 1, TNFa.

21. The tissue composition of any of claims 1-15, wherein the tissue composition has a maximum stiffness from 0.5 N/mm to 4 N/mm.

22. The tissue composition of any of claims 1-16, wherein the tissue composition has a tensile strength from 0.04 to 0.10 MPa.

23. The tissue composition of any of claims 1-17, wherein at least a portion of the tissue composition is absorbable.

24. The tissue composition of any of claims 1-17, wherein at least a portion of the tissue composition is non-absorbable.

25. The tissue composition of any of claims 1-19, wherein the scaffold has a pore size from 500 μm to 1200 μm.

26. The tissue composition of any of claims 1-20, wherein the seeded cells are stem cells, embryonic stem cells, embryonic stem cell-derived cells, induced pluripotent stem cell-derived cells, progenitor cells, cardiac cells, a skeletal muscle cells, smooth muscle cells, liver cells, pancreatic cells, lung cells, bone cells, umbilical cord cells, endothelial cells, central nervous system cells, gastrointestinal cells, endocrine cells, salivary cells, mesenchymal stem cells, fibroblast cells, or paracrine cells.

27. The tissue composition of any of claims 1-21, wherein the seeded cells are seeded as spheroids or in the form of a cell sheet, in the form of a gel, or in the form of a foam.

28. A tissue composition comprising:
a. a scaffold; and
b. ECM-generating cells disposed on or on and within the scaffold;
wherein after a cryopreservation-thaw cycle the tissue composition exhibits cell-cell communication.

29. A tissue composition comprising:
a. a scaffold; and
b. ECM-generating cells disposed on or on and within the scaffold;
wherein after a cryopreservation-thaw cycle the tissue composition maintains its physical integrity.

30. A tissue composition comprising:
a. a scaffold; and
b. ECM-generating cells disposed on or on and within the scaffold;
wherein after a cryopreservation-thaw cycle the tissue composition wherein after a cryopreservation-thaw cycle, cells of the tissue composition secrete one or a combination of a hormone, an enzyme, a growth factor, a cytokine, or an RNA.

31. A tissue composition comprising:
a. a scaffold; and
b. ECM-generating cells disposed on or on and within the scaffold;
wherein after a cryopreservation-thaw cycle the tissue composition exhibits synchronous contractions.

32. The tissue composition of claim 30, wherein the amount of hormone secreted is not less than 10% of an amount of hormone secreted by cells of the tissue composition prior to the cryopreservation-thaw cycle.

33. The tissue composition of claim 30, wherein the amount of hormone secreted is not less than 20% of an amount of hormone secreted by cells of the tissue composition prior to the cryopreservation-thaw cycle.

34. The tissue composition of claim 30, wherein the amount of hormone secreted is greater than an amount of hormone secreted by cells of the tissue composition prior to the cryopreservation-thaw cycle.

35. The tissue composition of claim 30, wherein the amount of enzyme secreted is not less than 10% of an amount of enzyme secreted by cells of the tissue composition prior to the cryopreservation-thaw cycle.

36. The tissue composition of claim 30, wherein the amount of enzyme secreted is not less than 20% of an amount of enzyme secreted by cells of the tissue composition prior to the cryopreservation-thaw cycle.

37. The tissue composition of claim 30, wherein the amount of enzyme secreted is greater than an amount of enzyme secreted by cells of the tissue composition prior to the cryopreservation-thaw cycle.

38. The tissue composition of claim 30, wherein the amount of growth factor secreted is not less than 10% of an amount of growth factor secreted by cells of the tissue composition prior to the cryopreservation-thaw cycle.

39. The tissue composition of claim 30, wherein the amount of growth factor secreted is not less than 20% of an amount of growth factor secreted by cells of the tissue composition prior to the cryopreservation-thaw cycle.

40. The tissue composition of claim 30, wherein the amount of growth factor secreted is greater than an amount of growth factor secreted by cells of the tissue composition prior to the cryopreservation-thaw cycle.

41. The tissue composition of claim 30, wherein the amount of cytokine secreted is not less than 10% of an amount of growth factor secreted by cells of the tissue composition prior to the cryopreservation-thaw cycle.

42. The tissue composition of claim 30, wherein the amount of cytokine secreted is not less than 20% of an amount of growth factor secreted by cells of the tissue composition prior to the cryopreservation-thaw cycle.

43. The tissue composition of claim 30, wherein the amount of cytokine secreted is greater than an amount of growth factor secreted by cells of the tissue composition prior to the cryopreservation-thaw cycle.

44. The tissue composition of claim 30, wherein the amount of RNA secreted is not less than 10% of an amount of growth factor secreted by cells of the tissue composition prior to the cryopreservation-thaw cycle.

45. The tissue composition of claim 30, wherein the amount of RNA secreted is not less than 20% of an amount of growth factor secreted by cells of the tissue composition prior to the cryopreservation-thaw cycle.

46. The tissue composition of claim 30, wherein the amount of RNA secreted is greater than an amount of growth factor secreted by cells of the tissue composition prior to the cryopreservation-thaw cycle.

47. The tissue composition of any of claims 30 and 38-48, wherein the growth factor is one or a combination of IGF-1, VEGF, FGF, HGF, G-SCF, or TGF-b, angiopoietin-1, TBG-b3, PDGF-A, EPO, HBEGF, TGFa, Angiogenein, Angiopoientin-2, Endothelial Growth Factor, Leptin, PDGF-BB, SPARC, IL-6, IL-8, Interferon-gamma, IL-1a, IL-1b, IL-6, IL-8, Monocyte chemotactic protein 1, TNFa.

48. The tissue composition of any of claims 28-47, wherein the tissue composition has a maximum stiffness from 0.5 N/mm to 4 N/mm.

49. The tissue composition of any of claims 28-48, wherein the tissue composition has a tensile strength from 0.04 to 0.10 MPa.

50. The tissue composition of any of claims 28-49, wherein at least a portion of the tissue composition is absorbable.

51. The tissue composition of any of claims 28-49, wherein the tissue composition is non-absorbable.

52. The tissue composition of any of claims 28-51, wherein the scaffold has a pore size from 500 μm to 1200 μm.

53. The tissue composition of any of claims 28-52, wherein the ECM-generating cells are live, dead, or a portion of the ECM-generating cells are dead.

54. The tissue composition of any of claims 28-53, wherein the ECM-generating cells are seeded on the scaffold first and subsequently produce the ECM.

55. The tissue composition of any of claims 28-53, wherein the ECM is deposited on the scaffold prior to seeding of the ECM-generating cells.

56. A tissue composition comprising:
a. a scaffold; and
b. extracellular matrix (ECM) material disposed on the scaffold or on and within the scaffold; and
c. ECM-generating cells disposed on or on and within the scaffold;
wherein after a cryopreservation-thaw cycle the tissue composition exhibits cell-cell communication.

57. A tissue composition comprising:
a. a scaffold; and
b. extracellular matrix (ECM) material disposed on the scaffold or on and within the scaffold; and
c. ECM-generating cells disposed on or on and within the scaffold;
wherein after a cryopreservation-thaw cycle the tissue composition maintains its physical integrity.

58. A tissue composition comprising:
a. a scaffold; and
b. extracellular matrix (ECM) material disposed on the scaffold or on and within the scaffold; and
c. ECM-generating cells disposed on or on and within the scaffold;
wherein after a cryopreservation-thaw cycle, cells of the tissue composition secrete one or a combination of a hormone, an enzyme, a growth factor, a cytokine, or an RNA.

59. A tissue composition comprising:
a. a scaffold; and
b. extracellular matrix (ECM) material disposed on the scaffold or on and within the scaffold; and
c. ECM-generating cells disposed on or on and within the scaffold;
wherein after a cryopreservation-thaw cycle the tissue composition exhibits synchronous contractions.

60. The tissue composition of claim 58, wherein the amount of hormone secreted is not less than 10% of an amount of hormone secreted by cells of the tissue composition prior to the cryopreservation-thaw cycle.

61. The tissue composition of claim 58, wherein the amount of hormone secreted is not less than 20% of an amount of hormone secreted by cells of the tissue composition prior to the cryopreservation-thaw cycle.

62. The tissue composition of claim 58, wherein the amount of hormone secreted is greater than an amount of hormone secreted by cells of the tissue composition prior to the cryopreservation-thaw cycle.

63. The tissue composition of claim 58, wherein the amount of enzyme secreted is not less than 10% of an amount of enzyme secreted by cells of the tissue composition prior to the cryopreservation-thaw cycle.

64. The tissue composition of claim 58, wherein the amount of enzyme secreted is not less than 20% of an amount of enzyme secreted by cells of the tissue composition prior to the cryopreservation-thaw cycle.

65. The tissue composition of claim 58, wherein the amount of enzyme secreted is greater than an amount of enzyme secreted by cells of the tissue composition prior to the cryopreservation-thaw cycle.

66. The tissue composition of claim 58, wherein the amount of growth factor secreted is not less than 10% of an amount of growth factor secreted by cells of the tissue composition prior to the cryopreservation-thaw cycle.

67. The tissue composition of claim 58, wherein the amount of growth factor secreted is not less than 20% of an amount of growth factor secreted by cells of the tissue composition prior to the cryopreservation-thaw cycle.

68. The tissue composition of claim 58, wherein the amount of growth factor secreted is greater than an amount of growth factor secreted by cells of the tissue composition prior to the cryopreservation-thaw cycle.

69. The tissue composition of claim 58, wherein the amount of cytokine secreted is not less than 10% of an amount of growth factor secreted by cells of the tissue composition prior to the cryopreservation-thaw cycle.

70. The tissue composition of claim 58, wherein the amount of cytokine secreted is not less than 20% of an amount of growth factor secreted by cells of the tissue composition prior to the cryopreservation-thaw cycle.

71. The tissue composition of claim 58, wherein the amount of cytokine secreted is greater than an amount of growth factor secreted by cells of the tissue composition prior to the cryopreservation-thaw cycle.

72. The tissue composition of claim 58, wherein the amount of RNA secreted is not less than 10% of an amount of growth factor secreted by cells of the tissue composition prior to the cryopreservation-thaw cycle.

73. The tissue composition of claim 58, wherein the amount of RNA secreted is not less than 20% of an amount of growth factor secreted by cells of the tissue composition prior to the cryopreservation-thaw cycle.

74. The tissue composition of claim 58, wherein the amount of RNA secreted is greater than an amount of growth factor secreted by cells of the tissue composition prior to the cryopreservation-thaw cycle.

75. The tissue composition of any of claims 58 and 66-68, wherein the growth factor is one or a combination of IGF-1, VEGF, FGF, HGF, G-SCF, or TGF-b, angiopoietin-1, TBG-b3, PDGF-A, EPO, HBEGF, TGFa, Angiogenein, Angiopoientin-2, Endothelial Growth Factor, Leptin, PDGF-BB, SPARC, IL-6, IL-8, Interferon-gamma, IL-1a, IL-1b, IL-6, IL-8, Monocyte chemotactic protein 1, TNFa.

76. The tissue composition of any of claims 56-75, wherein the tissue composition has a maximum stiffness from 0.6 N/mm to 4 N/mm.

77. The tissue composition of any of claims 56-76, wherein the tissue composition has a tensile strength from 0.04 to 0.10 MPa.

78. The tissue composition of any of claims 56-77, wherein at least a portion of the tissue composition is absorbable.

79. The tissue composition of any of claims 56-77, wherein the tissue composition is non-absorbable.

80. The tissue composition of any of claims 56-79, wherein the scaffold has a pore size from 500 μm to 1200 μm.

81. The tissue composition of any of claims 56-80, wherein the ECM-generating cells are live, dead, or a portion of the ECM-generating cells are dead.

82. The tissue composition of any of claims 56-81, wherein the ECM-generating cells are seeded on the scaffold first and subsequently produce the ECM.

83. The tissue composition of any of claims 56-81, wherein the ECM is deposited on the scaffold prior to seeding of the ECM-generating cells.

84. A tissue composition comprising:
a. a scaffold; and
b. extracellular matrix (ECM) material disposed on the scaffold or on and within the scaffold; and
c. seeded cells in or in and on the ECM;
wherein after a cryopreservation-thaw cycle the tissue composition exhibits cell-cell communication.

85. A tissue composition comprising:
a. a scaffold; and
b. extracellular matrix (ECM) material disposed on the scaffold or on and within the scaffold; and
c. seeded cells in or in and on the ECM;
wherein after a cryopreservation-thaw cycle the tissue composition maintains its physical integrity.

86. A tissue composition comprising:
a. a scaffold; and
b. extracellular matrix (ECM) material disposed on the scaffold or on and within the scaffold; and
c. seeded cells in or in and on the ECM;
wherein after a cryopreservation-thaw cycle, cells of the tissue composition secrete one or a combination of a hormone, an enzyme, a growth factor, a cytokine, or an RNA.

87. A tissue composition comprising:
a. a scaffold; and
b. extracellular matrix (ECM) material disposed on the scaffold or on and within the scaffold; and
c. seeded cells in or in and on the ECM;
wherein after a cryopreservation-thaw cycle the tissue composition exhibits synchronous contractions.

88. The tissue composition of claim 86, wherein the amount of hormone secreted is not less than 10% of an amount of hormone secreted by cells of the tissue composition prior to the cryopreservation-thaw cycle.

89. The tissue composition of claim 86, wherein the amount of hormone secreted is not less than 20% of an amount of hormone secreted by cells of the tissue composition prior to the cryopreservation-thaw cycle.

90. The tissue composition of claim 86, wherein the amount of hormone secreted is greater than an amount of hormone secreted by cells of the tissue composition prior to the cryopreservation-thaw cycle.

91. The tissue composition of claim 86, wherein the amount of enzyme secreted is not less than 10% of an amount of enzyme secreted by cells of the tissue composition prior to the cryopreservation-thaw cycle.

92. The tissue composition of claim 86, wherein the amount of enzyme secreted is not less than 20% of an amount of enzyme secreted by cells of the tissue composition prior to the cryopreservation-thaw cycle.

93. The tissue composition of claim 86, wherein the amount of enzyme secreted is greater than an amount of enzyme secreted by cells of the tissue composition prior to the cryopreservation-thaw cycle.

94. The tissue composition of claim 86, wherein the amount of growth factor secreted is not less than 10% of an amount of growth factor secreted by cells of the tissue composition prior to the cryopreservation-thaw cycle.

95. The tissue composition of claim 86, wherein the amount of growth factor secreted is not less than 20% of an amount of growth factor secreted by cells of the tissue composition prior to the cryopreservation-thaw cycle.

96. The tissue composition of claim 86, wherein the amount of growth factor secreted is greater than an amount of growth factor secreted by cells of the tissue composition prior to the cryopreservation-thaw cycle.

97. The tissue composition of claim 86, wherein the amount of cytokine secreted is not less than 10% of an amount of growth factor secreted by cells of the tissue composition prior to the cryopreservation-thaw cycle.

98. The tissue composition of claim 86, wherein the amount of cytokine secreted is not less than 20% of an amount of growth factor secreted by cells of the tissue composition prior to the cryopreservation-thaw cycle.

99. The tissue composition of claim 86, wherein the amount of cytokine secreted is greater than an amount of growth factor secreted by cells of the tissue composition prior to the cryopreservation-thaw cycle.

100. The tissue composition of claim 86, wherein the amount of RNA secreted is not less than 10% of an amount of growth factor secreted by cells of the tissue composition prior to the cryopreservation-thaw cycle.

101. The tissue composition of claim 86, wherein the amount of RNA secreted is not less than 20% of an amount of growth factor secreted by cells of the tissue composition prior to the cryopreservation-thaw cycle.

102. The tissue composition of claim 86, wherein the amount of RNA secreted is greater than an amount of growth factor secreted by cells of the tissue composition prior to the cryopreservation-thaw cycle.

103. The tissue composition of any of claims 86 and 94-96, wherein the growth factor is one or a combination of IGF-1, VEGF, FGF, HGF, G-SCF, or TGF-b, angiopoietin-1, TBG-b3, PDGF-A, EPO, HBEGF, TGFa, Angiogenein, Angiopoientin-2, Endothelial Growth Factor, Leptin, PDGF-BB, SPARC, IL-6, IL-8, Interferon-gamma, IL-1a, IL-1b, IL-6, IL-8, Monocyte chemotactic protein 1, TNFa.

104. The tissue composition of any of claims 84-103, wherein the tissue composition has a maximum stiffness from 0.5 N/mm to 4 N/mm.

105. The tissue composition of any of claims 84-104, wherein the tissue composition has a tensile strength from 0.04 to 0.10 MPa.

106. The tissue composition of any of claims 84-105, wherein at least a portion of the tissue composition is absorbable.

107. The tissue composition of any of claims 84-105, wherein the tissue composition is non-absorbable.

108. The tissue composition of any of claims 84-107, wherein the scaffold has a pore size from 500 μm to 1200 μm.

109. The tissue composition of any of claims 84-108, wherein the seeded cells are stem cells, embryonic stem cells, embryonic stem cell-derived cells, induced pluripotent stem cell-derived cells, progenitor cells, cardiac cells, a skeletal muscle cells, smooth muscle cells, liver cells, pancreatic cells, lung cells, bone cells, umbilical cord cells, endothelial cells, central nervous system cells, gastrointestinal cells, endocrine cells, salivary cells, mesenchymal stem cells, fibroblast cells, or paracrine cells.

110. The tissue composition of any of claims 84-109, wherein the seeded cells are seeded as spheroids or in the form of a cell sheet, in the form of a gel, or in the form of a foam.

111. A workflow system for an engineered tissue composition, said workflow system comprising:
a. culturing the engineered tissue composition;
b. cryopreserving the engineered tissue composition; and
c. reconstituting the engineered, tissue composition, wherein the engineered tissue composition is functional after a cryopreservation-thaw cycle;
wherein the system is a closed system.

112. The workflow system of claim 111, wherein the system provides a sterile environment for culturing, cryopreserving, and reconstituting the engineered tissue composition.

113. The workflow system of any of claims 111-112, wherein the engineered tissue composition is an engineered tissue composition according to any of claims 1-110.

114. A method of enhancing a function of an engineered tissue composition, said method comprising: cryopreserving an engineered tissue composition according to any of claims 1-110; and reconstituting the engineered tissue composition, wherein the engineered tissue composition has enhanced function after a cryopreservation-thaw cycle.

115. The method of claim 114, wherein the enhanced function is enhanced secretion of an enzyme, hormone, cytokine, RNA, or growth factor.

116. A method of enhancing a function of an engineered tissue composition, said method comprising: chilling an engineered tissue composition according to any of claims 1-110 to a temperature from −196° C. to less than 37° C.; and reconstituting the engineered tissue composition, wherein the engineered tissue composition has enhanced function after being chilled.

117. The method of claim 115, wherein the enhanced function is enhanced secretion of an enzyme, hormone, cytokine, RNA, growth factor, etc.
